(19) United States
(12) Patent Application Publication
(10) Pub. No.: US 2014/0255984 A1
(43) Pub. Date: Sep. 11, 2014

(54) MICRO-ORGANISM THREAT DETECTION

(71) Applicant: Rosemary Katherine Cameron Sharpin, Auckland (NZ)

(72) Inventor: Rosemary Katherine Cameron Sharpin, Auckland (NZ)

(73) Assignee: ZYZEBA TESTING LIMITED, Auckland (NZ)

(21) Appl. No.: 14/128,940

(22) PCT Filed: Jun. 21, 2012
(86) PCT No.: PCT/NZ2012/000103

§ 371 Date: May 16, 2014

(30) Foreign Application Priority Data
Jun. 22, 2011 (NZ) 593660

(51) Int. Cl. C12Q 1/04; C12Q 1/10

(52) U.S. Cl. CPC C12Q 1/04; C12Q 1/10
USPC 435/38; 435/288.7; 435/34

(57) ABSTRACT

A method of tracking and predicting the spread of micro-organisms in an environment relies on the optical detection of the growth of micro-organisms within an analyte containing a growth medium for the micro-organism(s) to be detected. Standardisation of the containers and growth medium for each micro-organism allows rapid detection of the growth pattern of particular organisms and the implementation of a regime of remote self-reporting of results from portable incubator units (1201) in the field which include a memory (1206) to store the results, a GPS unit (1204) to provide location information and a transmitter (1203) to send the results to a central plotting organisation allowing rapid tracking and prediction of the spread of environmentally borne organisms.

### FIELD OF THE INVENTION

The invention generally relates to the inference of threats from the collation of results of detected micro-organisms across an extended area, to the culture and analysis of micro-organisms in order to determine the threat, both in the laboratory and in the field, and the collation of results from multiple instances of analysis to allow the drawing of the inference.

More particularly the invention relates to the detection and enumeration of multiple differing micro-organisms from changes in the colour of a culture medium in which the organisms are immersed, the tracking of populations of these microbiological organisms in certain locations and tracking of the occurrence and spread or decline of spatial populations of microbiological organisms.

Additionally the invention relates to the standardisation of a growth medium and adjuvants for micro-organisms in an analyte, and the medium to render such an analyte biologically inert after analysis.

More particularly the invention relates to the provision of a portable relocatable device for culturing micro-organisms, analysing the results and reporting these to a remote point for collation of data.

### BACKGROUND OF THE INVENTION

Dye reduction tests are known but are not considered to be reliable indicators of the type of microorganism or the quantity present. They provide a rough guide to indicate the presence or absence of bacteria.

The following is an extract from Atherton, H. V. and Newlander, J. A. 1977 Chemistry and Testing of Dairy Products. 4th Edn. AVI, Westport, Conn. cited at http://www.foodsci.uoguelph.ca/dairyedu/resazurin.html

“The methylene blue reduction test is based on the fact that the colour imparted to milk by the addition of a dye such as methylene blue will disappear more or less quickly. The removal of the oxygen from milk and the formation of reducing substances during bacterial metabolism causes the colour to disappear. The agencies responsible for the oxygen consumption are the bacteria. Though certain species of bacteria have considerably more influence than others, it is generally assumed that the greater the number of bacteria in milk, the quicker will the oxygen be consumed, and in turn the sooner will the colour disappear. Thus, the time of reduction is taken as a measure of the number of organisms in milk although actually it is likely that it is more truly a measure of the total metabolic reactions proceeding at the cell surface of the bacteria.

The resazurin test is conducted similar to the methylene blue reduction test with the judgment of quality based either on the colour produced after a stated period of incubation or on the time required to reduce the dye to a given end-point. The resazurin test may be a valuable time saving tool if properly conducted and intelligently interpreted, but should be supplemented by microscopic examination.

Results on the reliability of the resazurin tests are conflicting. One study in comparing the resazurin test with the Breed microscopic method on 235 samples found the test reliable. Other reports state that the resazurin test is an unreliable index of bacteriological quality in milk. A major criticism of the method is that the resazurin reduction time of refrigerated bottled milk at either 20° or 37° C. is much too long to be of any value in evaluating bacteriological spoilage of stored milk.

Standard Methods notes that under no circumstances should results of either methylene blue or resazurin tests be reported in terms of bacterial numbers. The two dye reduction procedures are described in more detail in Chapter 15 of the Thirteenth Edition of Standard Methods compiled by the American Public Health Association.”

There have been many attempts to develop tests to identify the bacterial species or to determine the extent of contamination. Most such tests require samples to be couriered quickly (preferably in a chilled state) to a laboratory, where the samples are cultured for 24 to 48 hours (typically on agar plates) and the resulting cultures examined by microscope to determine the amount and type of bacteria present. Typical turn around times for such tests is 3 to 5 days, which is far too long to provide adequate warning of contamination in waterways or on beaches. Resulting in the closure of beaches long after the contamination has passed. The time delays in completing and reporting such tests for foodstuffs especially for shellfish, means that either the batches have to be recalled after dispatch or held in store for 5 days until clear test results have been received. Similarly lengthy bacteriological testing of poultry and of dairy products, among others, has enormous economic consequences. There is clearly a need for a far more rapid yet accurate testing system for the presence and type of bacteria so that any contamination can be dealt with promptly and the source of the contamination can be determined so that remedial action can be taken. This is especially so in food processing plants, but applies also to marine farms.

It is known to measure the occurrence of microbiological organisms such as coliform bacteria in the environment by taking samples of organisms, culturing the samples in a suitable medium and measuring the number of organisms in the medium after culturing. It is also known to repeatedly sample particular locations, for instance swimming beaches, to provide an indication of the continuing state of the current population of an organism of interest. In this way the level may be monitored on a more or less continuous basis.

It is known to provide portable analysis devices for analysis of chemical substances, such as gases in the air, trace contaminants in water and metal particles in oil. Such devices are generally distinguished by low power requirements allowing the use of small batteries.

Unfortunately current methods for the culture of micro-organisms requires that the culture be kept at a constant temperature for a period of at least 24 hours. Maintaining a culture at a constant temperature for such a long period in a location where the device has no mains power supply is highly dependent on the ambient temperature, in that if the ambient temperature varies much from the required culture temperature the device will require an impractical battery size to give the required endurance. Additionally some method of accurately detecting the micro-organism count is required.

Ever since the creation of instruments able to test a sample for the presence of an analyte of interest, a need for containers for aiding in the collection and testing of the samples has existed. Accordingly, sample containers have been made that permit a user to take a sample, bring the sample to the lab, and then either transfer the sample to another container for testing, or use the container directly in the testing process. Although these previously developed containers are adequate, the containers are not without their problems. Moreover, it has been found that existing containers often require excess handling by human hands, often requiring the opening and closing of the container multiple times during sampling and testing, which often leads to the introduction of contaminates into the sample, and needlessly exposes the users to potentially harmful substances and micro-organisms. Also, once the testing has been done, often the testing container and its contents must be properly disposed of as hazardous waste as the contents may still contain substances or micro-organisms that may be harmful to humans or the environment.

Examples of prior art tests can be found in the following documents:

Background Art

| Patent | Title/Synopsis |
|---|---|
| FR 2 831 182 | Bioreactor for the culture and monitoring of micro-organisms |
| JP 10 150976 | APPARATUS FOR DETECTING MICROORGANISM |
| JP 09 159671 | METHOD AND EQUIPMENT FOR MEASURING COMPONENTS DERIVED |
| | FROM MICROORGANISM |
| U.S. Pat. No. 5,501,959 | Antibiotic and cytotoxic drug susceptibility assays using resazurin |
| EP 0 301 699 | Methods and apparatus for determining the bioactivity of liquid biological mixtures |
| U.S. Pat. No. 6,597,450 | Automated Optical Reader for Nucleic Acid Assays |
| U.S. Pat. No. 4,551,766 | Optical reader |
| U.S. Pat. No. 3,504,376 | AUTOMATED CHEMICAL ANALYZER |
| U.S. Pat. No. 6,055,050 | Photometer and test sample holder for use therein, method and system |
| U.S. Pat. No. 6,395,537 | Double container device and method for detecting and |
| | enumerating microorganisms in a sample |
| U.S. Pat. No. 6,653,096 | Process challenge device and method |
| WO2004/060766 | MIXING DISPENSER |
| WO2001/069329 | CONTROL FOR AN INDUSTRIAL PROCESS USING |
| | ONE OR MORE MULTIDIMENSIONAL VARIABLES |
| U.S. Pat. No. 4,321,322 | Pulsed voltammetric detection of microorganisms |
| HU0500591 | METHOD OF DETECTING AND COUNTING MICRO-ORGANISM |
| | IN SOLID, LIQUID OR AERIFORM SUBSTANCES |
| U.S. Pat. No. 5,366,873 | Device and method for use in detecting microorganisms in a sample |
| U.S. Pat. No. 6,197,576 | Instrument for detection of microorganisms |
| US20060285539 | System and method for transmitting analysed data on a network |
| U.S. Pat. No. 6,465,242 | Portable incubator |
| WO2006/123946 | DISPENSING CLOSURE HAVING MEMBRANE |
| | OPENING DEVICE WITH CUTTING TEETH |
| U.S. Pat. No. 4,174,035 | Two-component container and package |
| U.S. Pat. No. 4,103,772 | Sealed container with frangible partition |
| WO2004/085278 | CLOSURE WITH PUSH TYPE OPENER |
| WO2002/074647 | A PUSH/PULL CLOSURE |
| WO1990/014288 | SPOUT FOR FLASKS AND SIMILAR RECEPTACLES, WITH A |
| | PIERCING ELEMENT FOR PIERCING A LID ON RECEPTACLE NECKS |
| U.S. Pat. No. 4,903,828 | Bottle closure cap for two-component packages |
| WO0027717 | DISCHARGE CAP FOR RELEASABLE TABLET |
| GB659553 | Drop-delivering bottles |
| DE4139784 | Bottle-type container with refill unit |
| NZ540021 | Dispensing closure having membrane opening device with cutting teeth |
| WO98/38104 | A PACKAGE FOR KEEPING PRODUCTS SEPARATE BEFORE USE |
| U.S. Pat. No. 6,098,795 | Device for adding a component to a package |
| WO03/106292 | A DRINK CONTAINER FOR COMBINING A POWDER WITH A LIQUID |
| WO99/24806 | METHOD FOR DETERMINING OPTIMALLY WEIGHTED WAVELET TRANSFORM |
| U.S. Pat. No. 4,757,916 | Unit allowing two products to be stored separately and to be simultaneously |
| U.S. Pat. No. 4,637,934 | Liquid container with integral opening apparatus |
| U.S. Pat. No. 6,059,443 | Method and system for storing and mixing two substances in a container |
| U.S. Pat. No. 5,984,141 | Beverage storage and mixing device |
| US2005/0218032 | Sterile cleaning kit |
| CA2176895 | Test pack with gelled contents and colour testing. |
| CA2199445 | Testing pack with separate adjuvant and deactivant. |
| DE4139784 | Growth bottle with insertable capsule. |
| GB659553 | Bottle with contents expressed by squeezing. |
| NZ541057 | MIXING DISPENSER |
| US2004-028608 | Swab testing of biological presence. |
| US2005-218032 | Bottle contains a second bottle with contents mixing into first. |
| U.S. Pat. No. 4,321,322 | Detecting growth curves of organisms by electrical conduction. |
| U.S. Pat. No. 4,406,547 | Automated consecutive reaction analyser |
| U.S. Pat. No. 4,637,934 | Infant bottle with puncturing of sealed outlet by internal mechanism. |
| U.S. Pat. No. 4,757,916 | Container dispenses two separated products at mixing. |
| U.S. Pat. No. 4,903,828 | Bottle closure for two component pack. |
| U.S. Pat. No. 4,925,789 | Indicating component for micro-organism. |
| U.S. Pat. No. 5,164,301 | Micro-organism indication by fluorescent dye. |
| U.S. Pat. No. 5,284,772 | Resealable test specimen bottle. |
| U.S. Pat. No. 5,393,662 | Coliform detection colour contrast test. |
| U.S. Pat. No. 5,411,867 | Coliform detection successive colour test. |
| U.S. Pat. No. 5,605,812 | Coliform detection by gel plate colour count. |
| U.S. Pat. No. 5,610,029 | Micro-organism growth medium with colour change. |
| U.S. Pat. No. 5,620,865 | Growth medium suppresses other than Enterococci. |
| U.S. Pat. No. 5,620,895 | Bag with multiple sample wells. |
| U.S. Pat. No. 5,827,675 | Bioluminescent ATP assay kit. |
| U.S. Pat. No. 5,965,453 | Beverage container mixes two beverages. |
| U.S. Pat. No. 5,984,141 | Beverage dispenser dispenses two beverages. |
| U.S. Pat. No. 6,055,050 | Photometer measure luminescence from micro-organism sample. |
| U.S. Pat. No. 6,059,443 | Mixing container with second substance in sealed capsule in neck. |
| U.S. Pat. No. 6,060,266 | Portable e coli culture kit with heater (chemical), |
| | growth curves, optical detection and culture deactivation. |
| U.S. Pat. No. 6,465,242 | Portable incubator - container has recess for heater. |
| U.S. Pat. No. 6,978,212 | Remote analysis and central reporting of analytes. |
| U.S. Pat. No. 6,984,500 | Portable culture kit with growth curves. |
| WO00/27717 | Container with vent passage. |
| WO03/106292 | infant bottle - figures show cuttable seal. |
| WO95/23026 | APPARATUS AND METHOD FOR QUANTIFICATION OF |
| | BIOLOGICAL MATERIAL IN A LIQUID SAMPLE |
| WO98/38104 | Bottle includes screw cap with rupturable capsule. |
| WO99/24086 | Multi-compartment flexible container. |
| | Seals between compartments ruptured by manipulation. |
| WO2004009756 | PROLIFERATION AND DELIVERY APPARATUS |
| U.S. Pat. No. 5,292,644 | Rapid process for detection coliform |
| U.S. Pat. No. 5,364,766 | Culture medium for rapid count of coliform bacteria |
| U.S. Pat. No. 5,817,475 | Automatic microbiological testing |
| U.S. Pat. No. 5,528,363 | Integrated device for instantaneous detection and identification of an entity |
| U.S. Pat. No. 5,003,611 | Method for detection of the presence of undesired microorganisms |
| U.S. Pat. No. 6,372,485 | Automated microbiological testing apparatus and method therefor |
| U.S. Pat. No. 6,849,422 | System and method for analysing antibiotic susceptibility of biological samples |
| JP3225484 | EXAMINATION OF MICROORGANISM, EXAMINATION OF |
| | NUMBER OF MICROORGANISM, TOOL FOR EXAMINING MICROORGANISM |
| U.S. Pat. No. 6,597,450 | Automated Optical Reader for Nucleic Acid Assays |
| US2008/0179331 | Dispensing Closure Having Membrane Opening Device With Cutting Teeth |
| U.S. Pat. No. 5,728,542 | Disposable test kit apparatus and method for bacteria |
| U.S. Pat. No. 7,828,141 | Container closure having a capsule inside it |

The Applicant has described a novel test machine and method based on machine detectable changes in the colour and transmissivity in a growth medium as a function of the number of micro-organisms initially present in the sample, as described in the applicants NZ patent 539210. Typically the growth medium is a liquid which contains a micro-organism growth promoting adjuvant and an indicator such as resazurin which is reduced by the action of the micro-organisms, thereby changing the colour of the growth medium. The colour changes are due to changes in the indicator, changes in other chemical molecules in the liquid, and changes in the reflection of light from particles, such as organisms or organism clumps, in the liquid. Turbidity may be one of the factors affecting the overall transmission of light through the medium.

While it is possible to measure the growth of organisms optically as in the above patent application it is not possible to differentiate between organisms if there is more than one organism in the sample. The growth curves for populations of two or more organisms form one result and observation does not give any indication of which organism predominates or is most important from the user's viewpoint.

In such circumstances it is normally necessary to resort to growth and inspection on agar plates in order to differentiate the micro-organisms.

Therefore a need exists for a solution to the problem of tracking populations of micro-organisms in one or more locations and predicting from the measurements the past and future populations of the micro-organisms.

There still remains the problem of tracking the occurrence of a micro-organism over an extended area, detecting the progression of the population both in terms of area and level of occurrence, and predicting the future population.

Further, if a population or incipient population is to be monitored over an extended physical area, the human effort required to do this is high, involving repeated measurement in many places.

It would be desirable to provide a portable device for in-situ analysis of micro-organisms and remote reporting to allow simple detection of biological contamination at remote sites, or the spread of organisms, as for instance in a “red tide”. It would then be possible to provide unskilled staff with the devices to carry into the field to be placed at a desired location, supplied with a sample to culture, and left to report the results of the culturing to a central point. This would allow the rapid delineation of a microbial threat and the relatively simple tracking of the area of its expansion or contraction.

This rapid deployment and return of results allows the creation of a method of providing a response to biological contamination in many foodstuff supply situations.

A number of such applications are listed below. All of these are industries or services which can occur within a physical catchment or series of catchments.

Each industry or service can be viewed in the same way by identifying each stage of the supply or production of a foodstuff at which biological contamination could take place and providing for testing at these stages. For instance with a dairy farm, the milking process, the transportation, the processing, the distribution and supply chain all provide opportunities for contamination. Just as the water in a large physical catchment flows through the landscape possibly degrading on its journey, so too does the milk from the farm to the consumer face similar biological challenges.

In each case a catchment for a food product is inter-related and affected by human intervention. In each of the supply services below a possible point of contamination is identified. At each of these points a contamination test could be made.

### 1. Agriculture

Stock water supplies (effluent contamination)
Enviro-effluent management
Irrigation systems
Poultry and egg production
Equine industry

### 2. Dairy Industry

Water quality onto the farm (check for upstream contamination)
Dairy production (wash down)
Cream test/Col test
Discharge stream check
Tanker pick up test
Production line testing (milk powder, butter, cream)
Non-bacterial testing (e.g. melamine)

### 3. Horticulture

Irrigation systems (internal, external)
Hydroponics cultivation systems
Produce cleaning and systems
Spraying contractors
Frost protection systems

### 4. Aquaculture

Fish and crustacean farm water quality
Fish and crustacean processing plants
Aquarium management
Fish feed manufactures
Discharge from waterways into sea

### 5. Hospitality

Commercial cleaning
Catering services
Fast food
Restaurant management
Hotels

### 6. Emergency Management

Commercial and residential flood servicing and restoration
Emergency service vehicles (potable water storage)
Hazardous substance management and control
Field hospital supplies
Epidemic and disease management

### 7. Travel

Holiday parks, camp grounds
Water supplies—wells, rivers
Health resorts—day spas
Cruise ships: supply, storage, preparation and delivery
Airlines: supply, storage, preparation and delivery
Rail travel: supply, storage, preparation and delivery
Sea freighters: supply, storage, preparation and delivery\
   8. Industrial, commercial, residential liability investigation
Water quality testing
Food safety testing
Insurance consultancy and liability
Risk management consultants
Contamination source/catchment issue investigation

### 9. Infrastructure

Water testing laboratories
Water carriers
Water storage tanks and pipe construction/cleaning
Commercial and domestic well drilling servicing
Leachate testing: contaminated land fill sites, dump sites, recycling centres, construction and erosion sites
Laboratory testing: all water samples and liquid washes
Drainage testing: contamination/infiltration
Effluent treatment plants including disposal and spreading
Water recycling and purification plants
Rural and urban potable water supply testing
Rural and urban recreational/environmental water testing

### 10. Commercial

Water bottling plants, including vending machines
Aseptic packaging specialists
Water-coolers: manufacturing/servicing/consumables
Water filtration and purification: Initial testing, unit servicing and consumables
Air condition cooling towers: Equipment testing and performance testing
Food canning plants
Bakeries
Soft drink, fruit juice, vineyards, breweries
House and building inspection services
Chemical manufacture
Ice making manufacturers
Refrigerator and cool store maintenance
Food machinery manufacturing and servicing
Meat and produce curing and preservation
Commercial water blasting/chemical was down
Commercial laundries (Thailand death of NZ girl example)
Pipe fabricators, lining and cleaning contractors
Plumbing contractors
Medical and health care clinics
Retirement and convalescent homes
Safety equipment and product stores
Sanitary hygiene services
Spa pool cleansers and maintenance
Swimming pool servicing

### 11. Recreation

Water craft: Personal and hire—storage and delivery of water from storage tanks
RV: Personal and hire—storage and delivery of water from storage tanks
Fresh Water recreational testing including waterways, rivers, lakes, ponds, fountains and public swimming pools
Salt Water recreational testing including beaches, estuaries and water ways
   12. Aid projects
On site evaluation of potable water quality
Performance monitoring of on-site potable water treatment and storage systems
Data provision for chemical dosing: remote water supplies

### 13. Military Applications

On site evaluation of potable water quality (bacterial and other)
Performance of on-site potable water treatment and storage systems
Data provision for chemical dosing—remote water facilities
Mess hall and kitchen management
Field kitchen management

### 14. Education

School swimming pools
School kitchens and food service areas
Curriculum based study for school in their communities—environmental and residential water supply monitoring

### 15. Professional Sporting and Cultural Groups

Potable water and liquid monitoring during event management
Marae based events (water quality issues)

### 16. Medical

Urinary tract infection
Fluid monitoring for stable results

### 17. Regulatory Authorities

Local, regional, central government agencies health and quality standards monitoring

### 18. Skiing Industry

Water and food based
Snow making operations.
   Each of these environments requires some method of providing results for tests of contamination with as little delay as possible. Therefore a need exists for a solution to the problem of how to provide a self-supporting micro-organism analysis device which on practical supplies of battery power will culture a sample for only long enough to provide a reliable result and report it to a remote point.

Accordingly, there exists a need for an improved testing container that has one or more of the following characteristics: reduces the potential of contaminates being introduced into the container during sampling or testing, reduces the potential that the contents of the container will cause harm to humans or the environment during or after sampling or testing is complete, is more reliable, is less expensive to manufacture, has less parts, is easier to use, and is more reliable.

The present invention provides a solution to this and other problems which offers advantages over the prior art or which will at least provide the public with a useful choice.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Within the specification reference is made to colour “frequencies” and colour “bands”. The reference to “frequency” is to the general frequency of light of a particular colour and to “band” as light extending over a range of frequencies but of one general colour.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein; this reference does not constitute an admission that any of these documents form part of the common general knowledge in the art, in New Zealand or in any other country.

### OBJECT OF THE INVENTION

It is an object of the invention to provide a testing method for aiding a user in the collection and testing of an environmental sample that ameliorates some of the disadvantages and limitations of the known art or at least provides the public with a useful choice.

It is a further object of the invention to provide a method of automatically carrying out tests of environmental samples and reporting them back with the possibility of collating continuing results to produce an indication of any threat from the environment.

It is a further object of the present invention to provide a solution to this and other problems which offers advantages over the prior art or which will at least provide the public with a useful choice.

### SUMMARY OF THE INVENTION

In a first aspect the invention provides a portable micro-organism detection apparatus comprising:
an incubator at least partially surrounding a container receiving space capable of receiving a rigid substantially transparent fluid container with at least one light path through the container
at least one light source mounted on or in an incubator capable of transmitting light into the fluid container
at least one light sensor mounted on or in the incubator and capable of detecting light of at least one colour which has passed through at least part of the fluid from the at least one light source
stored calibration information on a non-transient medium on the light changes over time measured in a similar container containing a sample of a known identified micro-organism
location detection means connected to or mounted on the incubator to provide location data of where the sample was taken
a micro-processor capable of controlling the operation of the incubator and the light source(s) and light sensor(s) and comparing, analyzing and storing the results, and receiving location data from the location detection means
stored calibration information on a non-transient medium on the light changes over time measured in a similar container containing a sample of a known identified micro-organism
the micro-processor having a comparator capable of detecting changes over time resulting from a sample in the rigid substantially transparent fluid container being incubated in the incubator, and comparing it with the stored calibration information to determine the presence or absence of a particular micro-organism
a data logger to store the results of the comparison and a date and time stamp and location information from the location detection means.

Preferably the incubator has a transmitter to send the results to a base station.

In another aspect the invention provides a method of detecting micro-organisms from changes in the colour of a growth medium in which a sample potentially containing micro-organisms is immersed, by sealing a sample from a location in a rigid substantially transparent fluid container with at least one light path through the container, placing the container in an incubator, transmitting light into the fluid container over time from at least one light source mounted on or in an incubator; detecting light of at least one colour which has passed through at least part of the fluid from the at least one light source by at least one light sensor mounted on or in the incubator, sending data from the at least one light sensor to a micro-processor having a comparator which analyses the light changes over time resulting from a sample in the rigid substantially transparent fluid container being incubated in the incubator, by comparing the detected light changes with stored calibration information to determine the presence or absence of at least one particular micro-organism; and stores the results of the comparison for the sample in a data logger together with location information and a date and time stamp for that sample.

Preferably the results are transmitted to a base station and stored in a database.

Preferably a plurality of samples is analyzed and the results transmitted to the base station for analysis by a computer.

Preferably rate of growth over time of the micro-organism(s) within the sample is assessed to enumerate the number of micro-organism(s) detected at the location.

Preferably the computer analyses the occurrence or spread or decline of spatial populations of microbiological organisms from the sample data and location and date time stamp of each sample.

Preferably the rate of growth over time is matched against stored calibration information for differing micro-organisms to determine the closest match.

Preferably the growth medium is optimised for E. coli.

Preferably the growth medium contains lactose, Casitone, NaCl, KCl, CaCl2, and MgCl2 as growth adjuvants.

In a further aspect the invention provides a method of detecting micro-organisms from changes in the colour of a standardised growth medium containing one or more indicator dyes in which a sample potentially containing micro-organisms is immersed, by sealing a sample from a location in a rigid substantially transparent fluid container with at least one light path through the container, placing the container in an incubator, transmitting light into the fluid container over time from at least one light source mounted on or in an incubator; detecting light of at least one colour which has passed through at least part of the fluid from the at least one light source by at least one light sensor mounted on or in the incubator: sending data from the at least one light sensor to a micro-processor having a comparator which analyses the light changes over time resulting from a sample in the rigid substantially transparent fluid container being incubated in the incubator, by comparing the detected light changes with stored calibration information of different micro-organisms which have been calibrated by incubating known micro-organisms in the same type of container with the same amount and type of growth medium and same type of indicator dyes to determine the closest match or matches and report the presence or absence of the different micro-organisms.

Preferably the results of the analysis of the sample are stored in a data logger together with location information and a date and time stamp for that sample.

Preferably the results are transmitted to a base station and stored in a database.

Preferably a plurality of samples is analyzed and the results transmitted to the base station for analysis by a computer.

Preferably rate of growth over time of the micro-organism(s) within the sample is assessed to enumerate the number of micro-organism(s) detected at the location.

Preferably the computer analyses the occurrence or spread or decline of spatial populations of microbiological organisms from the sample data and location and date time stamp of each sample.

Preferably the growth medium contains lactose and growth adjuvants.

In a yet further aspect the invention provides a method of detecting and enumerating micro-organisms from changes in the colour of a growth medium in which a sample potentially containing micro-organisms is immersed, the recording of the enumeration of these micro-organisms in known locations from this enumeration the tracking of the occurrence and spread or decline of spatial populations of microbiological organisms characterised in that the growth medium is initially sealed within a container in which the sample is deposited, that the growth medium is released into the sample, that at least the transparency of the growth medium is monitored optically with time and that an enumeration of at least one micro-organism dependent on the elapsed rate of growth of the organisms within the sample is reported for recording at a remote location.

Preferably the transparency is monitored over at least one colour band.

Preferably the transparency is monitored separately over several colour bands.

Preferably the turbidity is monitored in addition to the transparency.

Preferably the elapsed rate of growth is matched against that for differing micro-organisms to determine the closest match.

Preferably a comparison is made by normalizing a record of transparency of a sample with time and comparing the normalized record with those of known micro-organisms in the same growth medium under the same physical conditions.

Preferably the elapsed rate of growth is matched against multiple micro-organisms within the one sample if no match is found against any one of the micro-organisms.

Preferably the growth medium sealed in the container is optimised for a particular micro-organism.

Preferably the growth medium is optimised for E. coli.

Preferably the growth medium contains lactose, Casitone, NaCl, KCl, CaCl2, and MgCl2 as growth adjuvants.

Preferably Casitone is present at a concentration of between 0.1% and 1% by volume.

Preferably lactose is present at between 0.5% and 10% by volume.

Preferably the growth medium contains bile salts as an inhibitor of other non-specific micro-organisms.

Preferably the growth medium contains a redox dye indicator.

In another aspect the invention relates to a micro-organism enumerator including:
a container including a growth medium for at least one micro-organism sealed in an isolated manner within the container
a closable container opening allowing insertion of a sample potentially containing micro-organisms
a release mechanism allowing release of the sealed growth medium into the sample within the container
an incubator for the container, maintaining the container and contents at a desired temperature
the incubator including an optical source and at least one optical sensor connected to a monitor continuously measuring at least the transparency of the sample and growth medium within the container
an incubator recorder logging at least the transparency of the sample and growth medium with time
an incubator transparency matching comparator comparing the change in transparency of the sample and growth medium with time against a database of possible such changes and retrieving the most probable match
   an incubator reporter reporting to a remote location the most probable match.

Preferably the container also includes a sealed biocide, releasable into the sample and growth medium.

Preferably the release is manually accomplished.

Preferably the sample transparency is measured by light passing through the container.

Preferably the light is originated in the incubator and detected by optical sensors in the incubator.

Preferably the sample transparency is measured and compared for a single colour of light.

Preferably the sample transparency is measured and compared for multiple colours of light.

Preferably the sample turbidity is additionally measured and compared.

Preferably the container may contain a growth medium for selectively growing a single micro-organism genus or species.

Preferably the container may contain a growth medium for selectively inhibiting the growth of one or more micro-organism genera or species.

Preferably the container growth medium is standard among all containers targeting a specific micro-organism.

Preferably the incubator reporter may contain a radio transmitter reporting the probable match.

Preferably the incubator radio transmitter is a cell phone.

Preferably the incubator is portable and self-contained as regards power for at least one sample growth cycle.

Preferably the container is transparent in at least those areas adjacent and opposed to the optical sources of the incubator.

Preferably the container is additionally transparent in those areas laterally perpendicular to the optical sources of the incubator.

Preferably the incubator includes a GPS receiver allowing self-identification of the location of the unit.

In the specification we have made reference to “location detection system” or “location detection means”. The most practical way of achieving this at the present time is to use a GPS chip (a chip set that accesses the global positioning satellite system in order to provide coordinates of the location based on triangulation from the satellites). This may be an off the shelf GPS receiver which is connected to the microprocessor, or it may be a chip set which forms part of the microprocessor.

Alternatively, other location detection systems may be used. For example the portable incubator could include a display screen, and a set of stored maps, and it may allow the operator to enter the location manually on the map stored in the incubator, or the incubator may include the coordinates of particular sample sites, which have been predetermined, and which have been identified on the map, or identified in some other way, so that the operator can then take the sample from that particular predefined location, and identify it by suitable reference to the sample number of sample location.

Preferably the incubator allows input of a desired geographically referenced location.

Preferably such a geographically referenced location may be remotely input.

In a further exemplification the invention relates to a method of cumulating and displaying the results of analysis for specific micro-organisms within a physical area by providing within that area multiple micro-organism analysis units, culturing samples from a known location within the physical area in a micro-organism analysis unit, providing remote from the physical area a central location capable of receiving results from analysis units, receiving at the central location from remote analysis units the results of analyses and the location of the analysis sample, displaying the results on a micro-organism count versus time basis on a map of the physical area, identifying from a time/count reversion a sub-area of the area as the tentative origin of the increase in micro-organisms, relocating at least analysis units to locations within the identified sub-area and repeating the method until the tentative origin is confirmed.

Preferably the remote analysis units are units as referred to above.

Preferably the step of identifying a sub-area includes as inputs the physical factors affecting transfer and propagation of the specific micro-organism.

Preferably the identification of a tentative origin may include the step of providing to the remote analysis units a new location of selected remote analysis unit.

Preferably each remote analysis unit includes a GPS location identifying unit and an audio and visual output identifying the route to the new location.

Preferably the results received from the analysis units include the time of sample collection.

Preferably multiple samples are collected from the same location over time and the method includes comparing information from the same location at different times to measure the change in occurrence at a specific location with time.

Preferably the comparison reveals the bacterial occurrence at locations within a specified area over time.

Preferably the growth trend is depicted on a map in both space and time.

Preferably the growth trend includes an output of a predicted population.

In another aspect the invention provides a method of tracking bacterial occurrence by:
a) taking samples at related geographical locations and storing a machine readable identification of the geographic location and time of each sample collected,
b) analysing the samples to obtain information on the presence, absence, or level of occurrence of specific bacteria in a sample
c) transferring the information on each sample and its location in a machine readable form to a database
d) comparing database information on bacteria at each location to identify populations of bacteria over a geographical area.

Preferably each unit includes a GPS component capable of storing the GPS co-ordinates at the time of the sample collection.

Preferably each unit is set to store an identification of the person taking the sample.

Preferably each unit uses a standardised population growth test where the samples are each grown in a standardised nutrient solution in a defined sample container in each unit (where the unit acts as an incubator and an a light recording device to detect and compare changes in light transmission and reflection within the sample under test with reference samples for known populations the data of which is stored in each unit.

Preferably the information is transferred by radio transfer of a data stream.

In a further exemplification the invention consists in a portable analysis device capable of receiving an at least partially transparent culture container containing a culture medium and including: a culture medium temperature control capable of maintaining the culture medium at a specified temperature; an optical transmissivity measuring means capable of measuring the optical transmissivity of the culture container and medium and detecting changes in the transmissivity indicative of the growth of at least one micro-organism in the culture; a data logger storing data representative of the detected changes; a transmitter transmitting the logger data to a remote location; and a power source capable of powering the device to a either a desired culture end point where a significant level of a micro-organism to be detected is present or for a time beyond that required to reach that level where the micro-organism is not present.

Preferably the portable analysis device includes a location detecting system.

Preferably the location detecting system is a Global Positioning System receiver.

Preferably the transmitter transmitting logger data is a GPRS, CDMA or other cellphone protocol transmission.

Preferably the optical transmissivity is measured over at least two pathways through the medium.

Preferably the reflectivity of the medium is also measured.

Preferably the portable analysis device has a disengageable battery pack.

Preferably the culture container is contained within the device in an insulated surround.

Preferably the culture container may be heated or cooled within the surround.

In an alternative embodiment the invention consists in a method of culturing micro-organisms in a portable analysis device comprising placing into an at least partially transparent culture container a sample, a culture medium and an indicator of micro-organism growth, placing the container into a portable device capable of maintaining the container at a substantially constant temperature, measuring the optical transmissivity of the medium at at least one optical wavelength, from changes in transmissivity detecting the presence or absence of a micro-organism desired to be detected, storing information on the presence or absence of the micro-organism, reporting to a remote point the stored information and powering the portable analysis device from an internal power source capable of powering the device to a micro-organism detection end point.

### BRIEF DESCRIPTION OF THE DRAWINGS

FIG. 1 is a diagram of the implementation of multiple micro-organism measuring stations in an environment.

FIG. 2 is a diagram of a larger area and the detection of an initial population of an organism.

FIG. 3 is a diagram of the same area as FIG. 2 showing the placement of additional measuring stations or sampling stations as a response to the detection described with reference to FIG. 2.

FIG. 4 is a general perspective view of the top of an incubation and analysis module of the portable analysis device.

FIG. 5 is a top view of the top of FIG. 4 with the incubation compartment lid removed.

FIG. 6 is a perspective view of the power supply and communications module of the portable analysis device.

FIG. 7 is an upper perspective view of the power and communications module with the cover removed.

FIG. 8 is a bottom view of the power and communications module with the cover removed.

FIG. 9 is a front view of the assembled modules with the covers removed.

FIG. 10 is a left side view of the assembled modules with the covers removed.

FIG. 11 is an exploded view of the inner portion of the incubation module.

FIG. 12 is a flow diagram of one analysis process.

FIG. 13 is a view of growth curves for multiple different organisms.

FIG. 14 is a number of growth curves from a multiple sample plate.

FIG. 15 is a flow diagram of the process of matching growth curves for single or multiple organisms at a single light frequency.

FIG. 16 is a growth curve for a single organism at multiple colours by transmitted light.

FIG. 17 is a growth curve for a second single organism at multiple colours by reflected light.

FIG. 18 is a differential of some of the curves for FIGS. 16 and 17.

FIG. 19 is a flow diagram for detecting the organism by using the inflection points of growth curves.

FIG. 20 is a front elevation view of one embodiment of a container formed in accordance with the present invention, the container shown encased in a protective covering and prior to mixing.

FIG. 21 is an exploded cross-sectional view of the container of FIG. 20, the cross-sectional cut taken vertically through the centreline of the container and shown with the protective covering removed for clarity.

FIG. 22 is an assembled cross-sectional view of the container of FIG. 20.

FIG. 23 is a front elevation view of the container of FIG. 22 shown after a sample collection member has been removed from the container, used to collect a sample, and returned to the container.

FIG. 24 is a front elevation view of the container of FIG. 22 shown after a first barrier has been compromised allowing a liquid to be mixed with a reagent additive and the sample.

FIG. 25 is a front elevation view of the container of FIG. 22 shown during testing of the contents of the container in a testing apparatus; and

FIG. 26 is a front elevation view of the container of FIG. 22 shown after a second barrier has been compromised allowing a prophylactic additive to be mixed with the contents of the container.

FIG. 27 shows a diagram including the major contamination points in the perishable foodstuffs supply chain.

FIG. 28 shows a flow diagram of the process of predicting threats from changes in the results of environmental testing.

The illustrated embodiment of the present invention relates generally to a sample testing container used in aiding a user in the collection and/or testing of a sample.

### DETAILED DESCRIPTION OF THE INVENTION

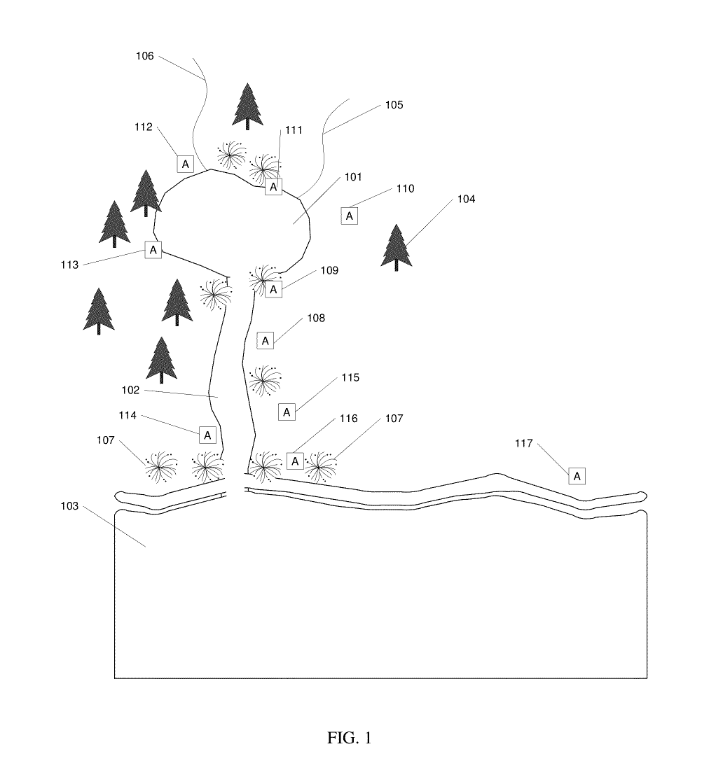

Referring now to FIG. 1 this shows an estuarine lake 101 fed from streams 105, 106 and connected to seashore 103 by an estuary 102. Grasslands 107 and trees 104 surround them all. Portable analysis units 108 to 117 are located throughout the area. These units have samples from the local environment and culture medium adjuvant together in a container placed in them when measurements of the current count of specific micro-organisms are required. The units culture the sample to the point where either a measure of the growth of the desired micro-organism is completed or no growth is found, and they then report the growth curve to a remote central location, typically by embedded cell phone modules.

The units may be recharged and read on a regular basis, for instance weekly, and can thus provide a measure of micro-organism numbers over time.

Readings will fluctuate with time, temperature, etc. but a persistent source of the target micro-organism could, for instance, be seen to be originating from site 112.

Readings from the analysis units are entered into a database, typically at a remote central location, and may be then correlated with environmental factors to allow determination of the source of a target micro-organism and prediction of it continued expansion or contraction. Thus in the example shown the current down the streams should be known as a function of previous, current and expected precipitation, the tidal flow in and out of the estuarine lake is known, the current along the coast is known, and the daily wind vectors are tracked, the historically expected wind vectors are known, the daily temperature is tracked, the historically expected temperatures are known, the forecast temperatures, wind and precipitation are known. An initial plotting at chosen locations provide only tentative identification of the source of the micro-organism and to identify the precise source of a particular micro-organism reversion predictions based on the data received may be used to indicate sub-areas within the main area to which analysis units should be moved for better resolution of the source.

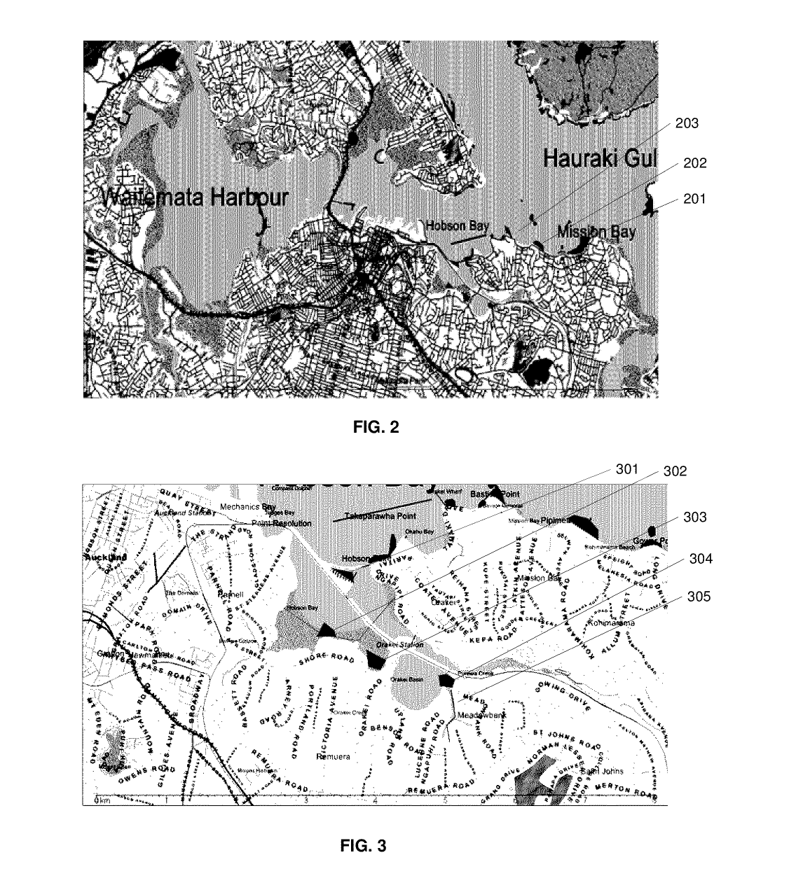

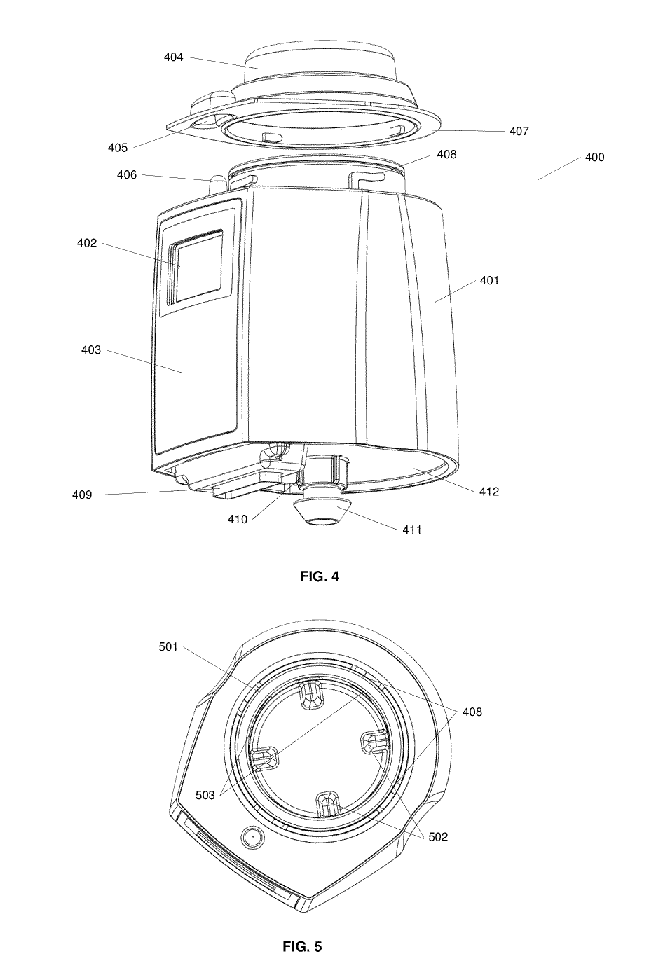

FIGS. 2 and 3 are real maps showing part of the Waitemata Harbour of Auckland, New Zealand. All place names are real place names to assist in explaining the invention.

FIG. 2 shows the simpler isolation of a source of contaminating micro-organisms, first detected at Mission Bay shown at 201, the bay being a popular bathing spot, and washed by the outgoing tide from the estuarine basin of the Waitemata Harbour. Further monitoring points 202 (Pipemea) and 203 (Bastion Point) were set up as a sub-area within the original overall area and the organism also detected there.

FIG. 3 shows the further successive tracking of the source as showing contamination at the Hobson Point marina (301), some contamination at Shore Road point (302) and the Orakei Station shoal (303) but more contamination at the Waitarua Creek outlet (304). Subsequent sampling up the stream by continued re-definition of the suspect area isolated the source to the Waitarua Road access point to the stream.

Normally such mapping is a slow process since obtaining sample culture results typically takes some 14 hours. Using the apparatus of New Zealand patent 539210 results may be obtained within 2 hours, drastically reducing the time taken to map the advance of a micro-organism and allowing one sample to be cultured while the apparatus culturing the sample is moved to the location at which the next sample is to be taken.

To make the best use of this rapid analysis each unit has an inbuilt GPS unit. This has two advantages. Firstly the precise geographical reference of the location of each unit can be automatically advised to the remote central location, and secondly each unit can be remotely programmed from the central location with its next preferred location. A relocation function allows the display screen to run a typical GPS route direction visual display with audio accompaniment to allow the remote unit bearer to be directed to the next location.

Referring now to FIG. 4 this shows an incubation module 400 which includes a weatherproof cover 401, a viewable LCD panel 402 showing the current status of any analysis, a touch sensitive keypad 403 and a lid 404 for an incubation compartment better seen in FIG. 5. Lid 404 has a recess 405 which interengages with location and rotation stop 406 on the top of the module body, and nubs 407 which interengage with slots 408 around the periphery of the incubation compartment. While a touch sensitive keypad is shown the cover may equally well have a removable portion giving access to a fold down keyboard which may be preferred by some users.

Lid 404 may be lowered into place and secured by rotation to form a weather tight seal with the top of the incubation compartment which will contain a standardised transparent container with a standardised analyte containing a sample from the environment. A cover (not shown) may be placed over the lid to provide additional weather protection.

On the base plate 412 of the module are a connector 409 for supplying power and output connections to the incubation module and a locating and locking projection 410 with a release washer 411 which mates with the power and communication module.

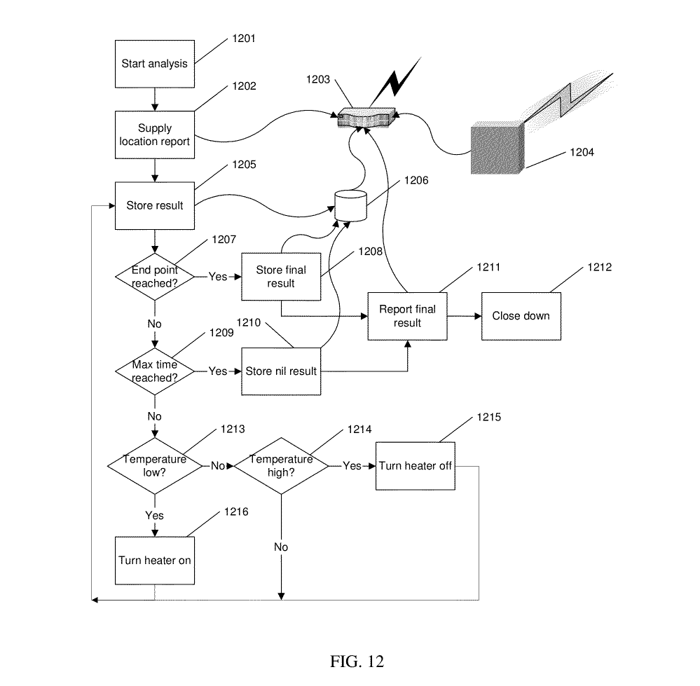

FIG. 5 shows a top view of the incubation module with the lid removed to show a removable spill tub 501, better seen in FIG. 12, having abutments 502 on which a culture medium container may rest and slots 503 which allow air circulation through the spill tub and also provide an aperture for the micro-organism detection optics. These apertures do not extend to the bottom of the tub so that spills from a culture medium container are captured without contaminating the remainder of the module.

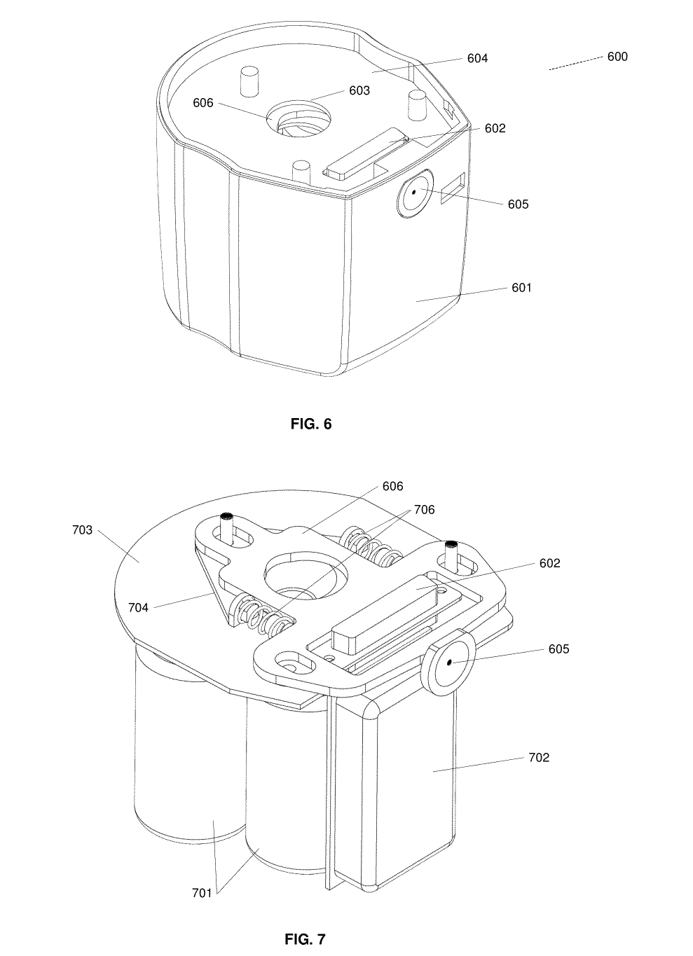

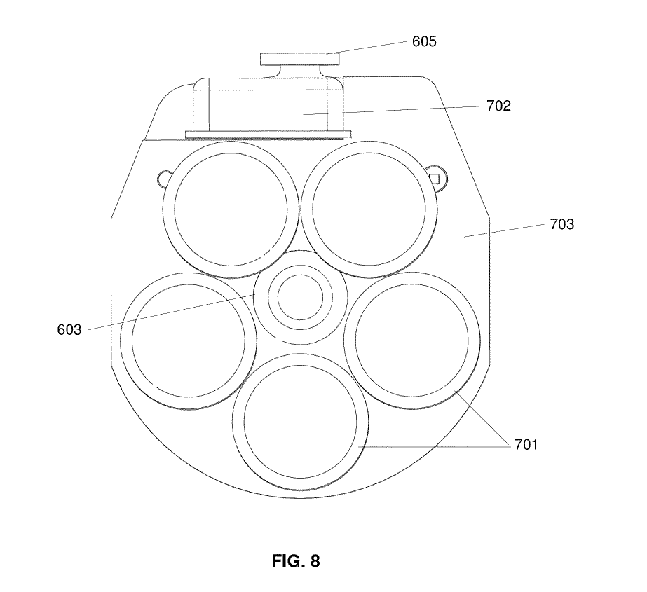

FIG. 6 shows the exterior of the power and communications module 600 which is disengagable from the remainder of the incubator and which has a weatherproof cover 601, a connector 602 mating with connector 409 of the incubator module, a top plate 604, a locating hole 603 in top plate 604 mating with projection 410 on the incubation module, and a release mechanism (FIG. 7) for the release plate 606 which is actuated by push button 605.

FIG. 7 shows the same module with the cover 601 and top plate 604 removed to show batteries 701, a modularised GPS receiver, data logger and communications unit 702 which can communicate with a remote point using an integrated cell phone unit, and a connector board 703 to which the batteries and connector 602 are mounted. Sandwiched between the connector board 703 and release plate 606 is the retention and release mechanism for the incubator module consisting of a spring location plate 704 and springs 706 which bias sliding plate 606 against projection 410 to capture release washer 411. Depressing push button 605 allows release of the incubation module from the power and communications module.

FIG. 8 shows a base view of the power and communications module where batteries 701 surround the central mating hole 603.

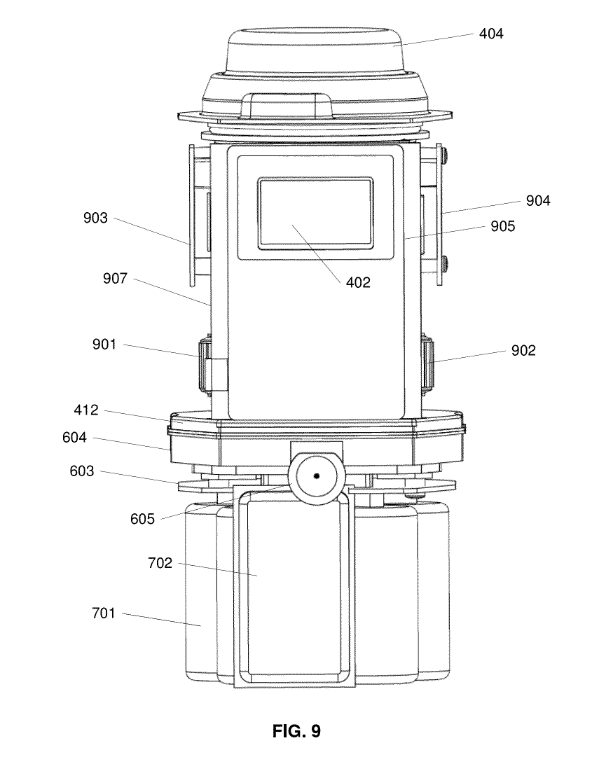

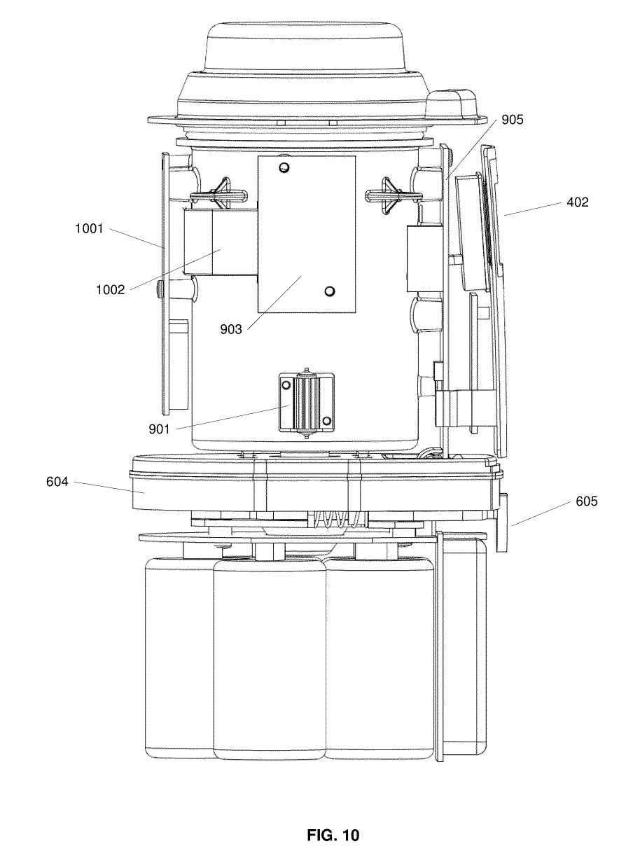

FIGS. 9 and 10 show respectively front, and side views of the two mated modules with the covers removed. Notable are heater units 901 and 902 which mount through plastics case 907 to an internal aluminium tube 501. Also shown are two circuit boards 903, 904. These boards mount respectively optical sources and sensors which cooperate with holes in the case 907 and gaps in the aluminium tube surrounding the central cavity so that light can be transmitted into and through the sample being cultured in a substantially transparent container in the central cavity.

The side view of FIG. 10 shows front and rear circuit boards 905 and 1006 which carry optical sensors for detecting scatter of the light from the source LEDs on side boards 904, 904 and the main processing circuitry for detecting the trend in light obscuration and scatter at various wavelengths and matching the scatter to one or more organisms.

The LED light sources may operate on different optical wavelengths, for instance white, red, green, yellow and infrared wavelengths. Each sensor may be filtered to respond only to a restricted waveband.

A ribbon cable 1002 interconnects the circuit boards including that carrying the touch sensitive keyboard and LCD display 102.

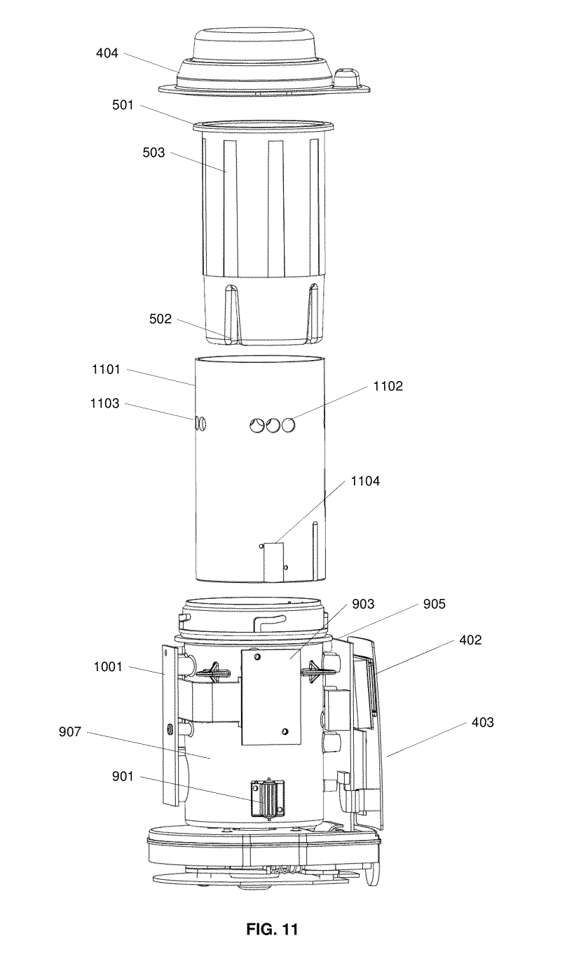

FIG. 11 shows an exploded view of the core components with spill bucket 501 having abutments 502 and slots 503 to allow passage of the light from the LEDs to the sensors. The bucket is contained within aluminium tube 1101 having holes 1102, 1103 for light from the light sources to the sensors. Heater 901 attaches to mount 1104 to provide for heat distribution throughout the aluminium tube 1101. Tube 1101 acts to maintain an even temperature throughout the central cavity while case 907 acts to insulate the cavity from the environment.

While the present example shows heaters it is equally possible to also provide Peltier effect cooling devices where the ambient temperature is expected to exceed the required incubation temperature.

FIG. 12 shows the process followed in analysing or enumerating a sample. Once the sample is in place and the analysis started at step 1201 an initial location report is made at 202 using the embedded cellphone module 1203 and a location obtained from a GPS module at 1204.

The GPS does not need to be on all the time and would normally be switched on only at the start and end of the analysis to provide an initial location and a confirming location for the final report, however it may be turned on at regular intervals so that any change in the desired location for the module can be remotely downloaded from a central location and used to prompt a transfer in the location of the incubation module.

The results of the location report are taken at 1205 and stored at 1206, normally using flash RAM (random access memory) to retain the data. At step 1205 also the illuminating LED's are switched on and a reading taken of the transmissivity or transparency and reflectivity of the sample at the requisite wavelengths. At 1207 it is determined whether the sample has reached an end point and identified the level of micro-organism presence in the original sample. If so the final result is stored at 1208, reported at 1211 via the remote communication from module 1203 and then the whole device is closed down at 1212.

If no end point is detected a check is made at 1209 to determine whether the longest possible time within which a result could be expected has expired and if so a nil result is stored at 1210, and again a final report and close down takes place.

After these checks the temperature in the sample chamber is checked and if at 1213 it is found to be too low the heaters 601, 602 are switched on at 1216. Similarly if the temperature is found at 1214 to be too high the heater is turned off at 1215, and then the measurement cycle repeats, typically at intervals of approximately one minute to conserve power.

In use a sample is placed in a transparent sample container and a culture medium containing a micro-organism sensitive indication component is added to the sample and agitated. Typically the indication component changes colour when the culture medium reaches a sufficient level of concentration of the target micro-organism. Such a component is resazurin, but others are well known.

The sample is then placed in an incubation and power/communication module assembly and the keyboard and LCD display are used to choose the required time/temperature program, which is specific to the micro-organism to be detected. The keyboard is then used to start the analysis, at which point the unit can be abandoned if necessary. It is programmed to report via the radio or cell phone link, giving its location as retrieved by the GPS, and its current settings. This information may be automatically entered into a remote database to allow future verification of the unit.

Data from the on-going development or otherwise of the micro-organism within the culture medium in terms of transparency, optical transmissivity or turbidity as an indicator of an elapsed rate of growth is logged to the incorporated data logger and eventually one of two things will happen. Either there will be no appreciable change in the growth medium indicator, indicating that the micro-organism is not present in the sample, or there will be a change in colour, transmissivity or reflectivity of the sample which can be interpreted as a measure of the existence of one or more micro-organisms. When the changes reach a desired end point the analysis unit informs the communications module which will send the logger data and an indication that it is closing down to sleep mode. In this latter state a cell phone call to the embedded cell phone number will allow the unit to be rewoken and remotely controlled if required.

Because the optical waveband sensing method gives results earlier than any other known method of determining the presence of specific types of micro-organisms it is feasible to provide a battery powered device remote from any power source which is usable by comparatively unskilled personnel, since all that is required is that a sample be properly loaded into the device, and the device be properly initialised.

While the version shown is not water resistant it is possible, for instance, to provide a freely floating version with batteries recharged by solar cells which will automatically suck a portion of the medium in which it is floating into a culture medium, incubate it to term and then provide results on its position at the time the culture was loaded so that a continuing record of micro-organism of interest is provided at current or tide driven locations. For continuing results each completed sample and culture run may be ended by dosing the culture medium with a biocide, flushing the medium from the culture chamber, cleaning the chamber with a flush of water and then placing more culture medium in the chamber and loading a sample.

While the version of the device shown is comparatively large because it is intended for a sample container which is easily handled manually it is feasible to miniaturise the sample, sample holder and incubation device, for instance by using nano-etching capabilities. This in turn results in a reduction in the size of the batteries required because less volume must be heated or cooled, resulting in a considerable reduction in size of the total device.

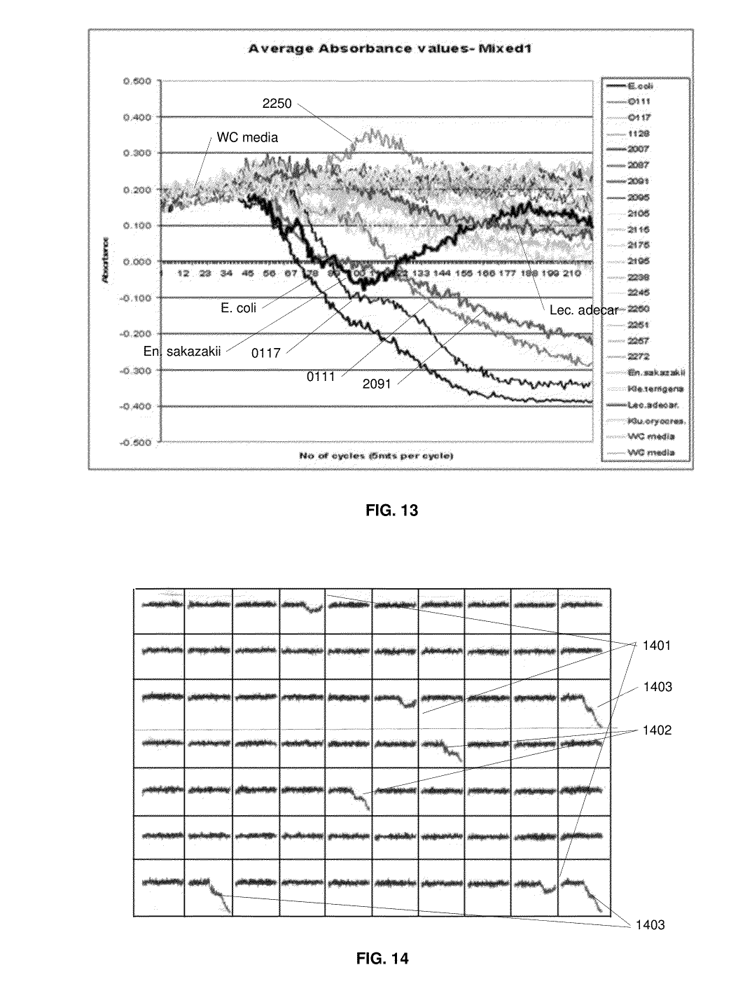

FIG. 13 shows the measurement of the growth curves of various micro-organisms in a liquid medium within a sample container. Typically the medium contains resazurin as an indicator and is incubated at a temperature of 37° C. The light reflected from a direct path through the medium and/or the colour transmissivity of the medium is measured at 5 minute intervals by the incubation module and the absorption or colour plotted until change appears limited. As shown in FIG. 13 this is after 215 5 minute cycles or about 18 hours. The particular light bandwidth for the curves shown is white light, and is effectively a measure of the amount of light detected at an angle to the light path through the medium. This light is dispersed by reflection from particles in the medium, which particles may be at least partially be the organisms being detected.

The figure shows superimposed growth curves for organisms such as E. coli, strains 0111, 0117, 2091, 2250 and for other micro-organisms such as En. sakarazakii and for control curves such as the medium without added micro-organisms as “WC media”. It should be noted that while many of these do not detectably react either at all or much in the particular growth medium or adjuvant used in the plot shown or with the particular frequency of light used, others provide very specific growth curves.

In FIG. 13 the growth curve for the medium (WC media) alone shows as a generally slowly upwardly trending line which peaks after some 16 hours at 37° C. and then slowly declines as the solution ages. Variation in the optical measurement process produces a certain amount of “noise” in the measurement process.

E. coli produces a rapid change in the medium colour from a time which is dependent on the initial count of organisms in the medium, but which in the example shown is approximately 4 hours onwards. The media then slowly clears until it stabilises after the 14 hour mark. The various E. coli show very similar curves, E. coli 2091, for instance, shows a similar curve initially, but after an initial growth spurt demonstrates a plateau of stability in medium colour before again continuing a regular change in turbidity. E. coli 0111 also shows a later onset, but here an initial slow change is followed by a period of more rapid change before slowly declining. En. Sakazakii, by contrast, shows an initial gradient close to that of E. coli before illustrating a notable upward gradient and then an eventual downward curve.

A large number of micro-organisms show no particular reaction in the medium illustrated and the light band illustrated, but these show different growth patterns in other media, at other temperatures and in differing light bands. The difference is sufficient that it can be said that there is some medium, temperature and light band which will allow almost any micro-organism to be differentiated from any other.

FIG. 14 shows growth curves as cumulated from a 70 cell plate reader, taken at 5 minute intervals and compressed so that a growth curve such as one of those in FIG. 1 is compressed into single cell. These show examples of a medium which has been diluted to the point where a single plate cell may or may not contain a single organism, and is very unlikely to contain more than two. Each of the growth curves can be resolved as a combination of one, two or more other curves for known organisms. It can be seen that most of the cells contain no organisms, but at 1401 are indicated three of the cells which contain one type of organism providing an identifiable growth curve as identified by the transmission through the cell. Two other cells 1402 show a different growth pattern and contain yet another organism, and three cells 1403 contain yet another growth pattern. This latter pattern may be due to either different organisms, but is more likely to represent cells in which two temporarily adhered organisms of the type shown in cells 1402 were located. The multiple organisms provide a faster growth curve, although the shape of the curve remains easily recognizable. It can be seen that any attempt to provide a bulk measure of mixed organisms must cope with growth curves which will differ in dependence on the number of organisms of each type in the medium.

To compare the growth curve with those taken from known organisms the start point of the curve is first regularised, i.e. the point at which any growth curve starts is detected since this may vary with number of organisms initially present. The new curve is then scaled along the horizontal axis (time) for the best fit (on the assumption that the temperatures or numbers of organisms may have been different) and then scaled on the vertical axis (quantity) for the best fit (on the assumption that the growth medium may not be identical). This process may be recursive and will provide as an end result a measurement of the degree of match of the body and end point of the compared growth curves. All of the available growth curves are matched against the new growth curve for a best fit and the closeness of the fit recorded.

If only a single organism is present it may be possible to get a very close match for at least one of the stored growth curves, however if more than one organism is present in the sample and grows in the medium the match will not be close and a second step takes place.

Taking the closest match to the current curve a second growth curve of known organisms is combined with the first curve to provide a combined growth curve. Again the curve from the second is scaled both on the time scale and on the quantity scale for best fit and a multitude of comparisons made from the stored growth curves to determine the overall best fit. If this is not within an acceptable range of fit (normally held as a difference in area of the current and the matching curves) the process may be repeated with the next most likely fit.

Eventually either one or more matches within the required standards are found, and a report of the best matches within the acceptable parameters are provided, with an indication of which is best and therefore most likely, or otherwise a failure report will issue, and a match by the dilution process which produced FIG. 14 may be required. Where the process is successful the calculations carried out will show the organisms found and also the concentration of organisms which were originally in the medium.

The comparison process may be carried out on a computer, either with the aid of a GUI interface and user assistance with the scaling, or totally under software control, preferably using “fuzzy match” algorithms in known manner to determine the best match.

In some instances simply the initial slope of the growth curve may be sufficient to allow classification. This is particularly so where, while there may be multiple organisms in the medium, only one of them is required to be identified. Thus the comparatively distinctive initial growth curve for E. coli can be distinguished early and unless the other organisms in the medium must be identified an indication of the presence of E. coli can easily be provided.

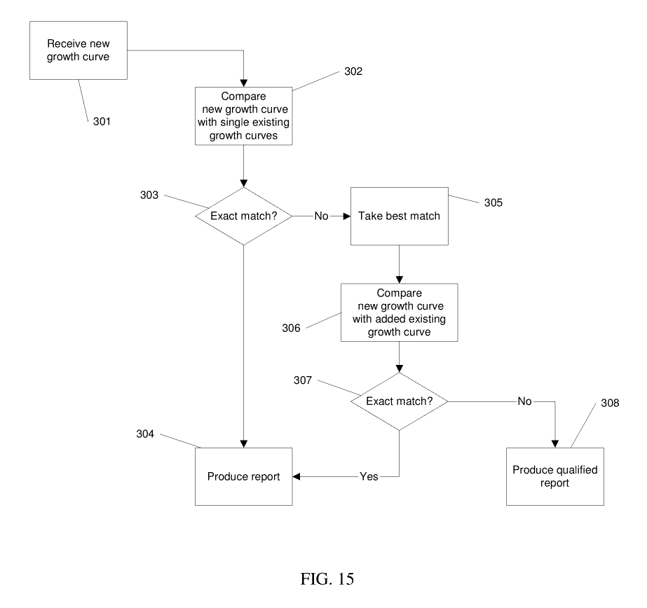

FIG. 15 shows a method of achieving such a match where a new growth curve to be matched is received at 1501, at 1502 this curve is compared with a single existing growth curve, with selection of the start point and scale of the curve. The comparison is carried out against all existing growth curves. If an exact match is detected at 1503 a report is produced at 1504, however if an exact match is not present then the process continues by taking at 1505 what is seen as the best match and adding single growth curves of other micro-organisms at 1506 in an attempt to achieve an exact match. If one of the attempts produces an exact match at 1507 a report is produced at 1504, otherwise after all possible matches has failed a qualified report of the best matches is produced at 1508. The matches are carried out by mathematical computation with either line or area measurement to indicate the “best fit” to the curves.

While the process as described above assumes a single growth curve the measurements may be made at multiple different bandwidths of light, and the matching process may occur for all of the bandwidths used in the process or for combinations of them. This may be required because the growth curves for differing wavelengths may be very different for some micro-organisms and this allows them to be confirmed or eliminated as being present in the measured sample. Similarly even if a measurement at a single band is made that band may be chosen to best show the organisms which are being preferably sought.

Because the detection of organism growth is optical and by either transmitted light or reflected light it is necessary that the container for the organisms and the medium or analyte is consistent as far as distortion of the optical path and absorption of the various wavelengths concerned. Thus the container should be transparent at the wavelengths concerned and the area through which the optical beams pass should have consistently shaped walls which do not appreciably distort in normal use. The container should be packaged such that the area through which the optical beams pass is not easily contaminated on the outside by the user. At the same time the containers are single use items, so cost is a factor, and hence polyethylene terephthalate with a polyvinyl alcohol coating to reduce oxygen absorption is used. An alternate choice may be an acrylic (styrene-acrylonitrile) container.

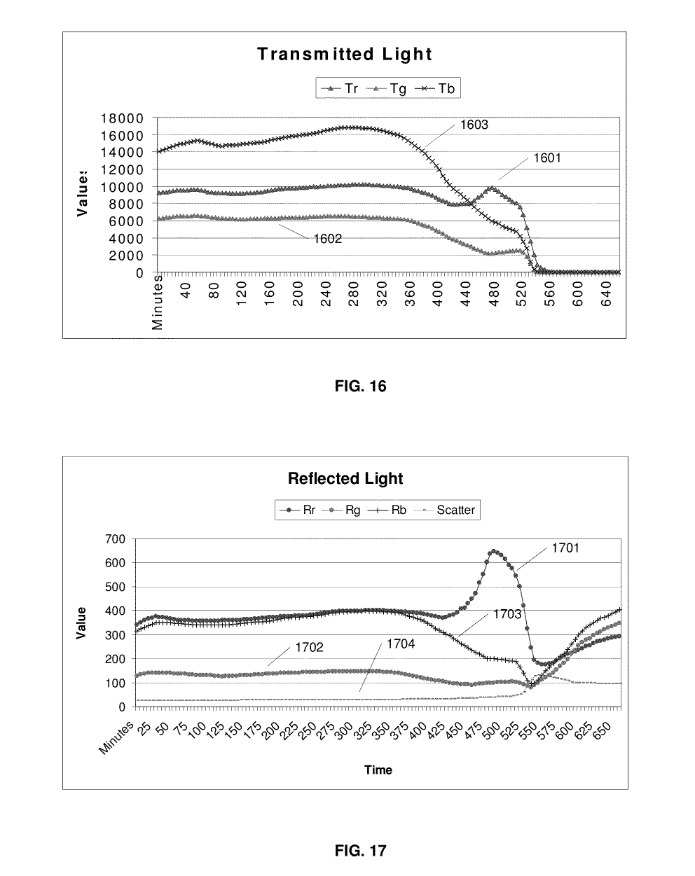

To this end FIG. 16 shows the transmission curves for one culture containing E. coli organisms in Resazurin while FIG. 17 shows the reflectance curves for the same culture. Also included in FIG. 17 is the amount of scatter for the culture, which is a measure of the light scattered from the light source. This may act as an indicator of the turbidity of the culture.

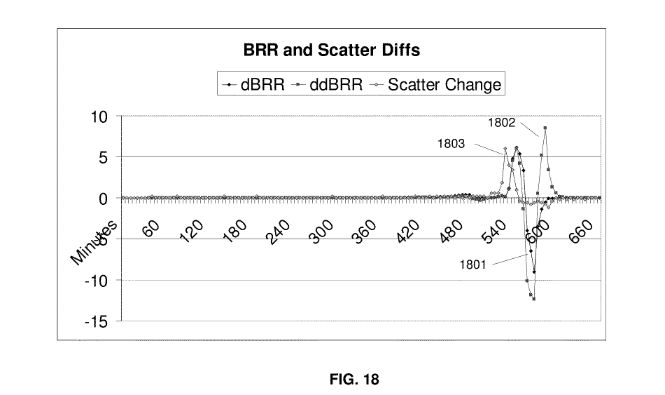

FIG. 16 shows the transmitted light in the red 1601, green 1602 and blue 1603 bands as measured for a particular culture, FIG. 17 the reflected light in red 1701, green 1702, blue 1703 and the scatter 1704 as measured at 90 degrees to the light path for the same culture, FIG. 18 shows the first differential for the combined (blue-red)/red light 1801, the second differential of this at 1802 and the differential for the scattered light 1704 at 1803.

It can be seen that over the period from 360 to 540 minutes after the start of the culture period large changes take place in both the turbidity and colour of the culture medium. To extract repeatably detectable events from these curves representing the growth of organisms within the culture a sequence of changes uniquely identifying a particular organism must be detected. In the culture growth pattern shown a series of events may be identified as:

A change in the ratio of blue to red colour (since these are the indicator colours for Resazurin).
A change in the turbidity of the culture.

Within these changes certain critical points can be mathematically identified, and in particular the inflexion points of the growth curves, as more clearly shown by the first and second differentials, are useful. One such set of critical points is identifiable by the sequence:
(a) The point at which the change (i.e. the first differential) in the ratio of ((transmitted blue−transmitted red)/transmitted red) goes appreciably negative.
(b) The point following this at which the second differential of the cumulated changes in the above reaches an appreciable value.
(c) The point after this at which the second differential of the cumulated changes goes negative and then appreciably positive again.
(d) The point after this at which a 3 point moving average of the change in the scatter goes appreciably positive.
(e) If these criteria are met the organism can be positively identified as E. coli and the level of contamination can be read as the scatter value at the time point (a).

This equates to: detecting the existence of a negative slope on the normalised blue−red comparison, then detecting the next upward swing in the comparison, then detecting a rise in the scatter detected after the first point detected. If these requirements are met then E. coli are present and predominant in the culture.

This pattern can be followed for other organisms, namely a first colour set by the indicator in use, a final colour set by the indicator in use, an initial turbidity in the culture, a final turbidity in the culture. There will be a distinctive hue change event, normally gradual but not necessarily so. Portions of the hue change will exhibit variations which are of a constant pattern for that organism. In addition to this, as the cultured organism grows the turbidity of the medium will increase, however this increase will not be at a linear rate, rather, as the organism grows the saturation curve will exhibit changes which may be rapid and which may reverse. This is currently thought to be due to stages of growth in the organism in which some organisms tend to group or clump, and in so doing temporarily reduce the turbidity.

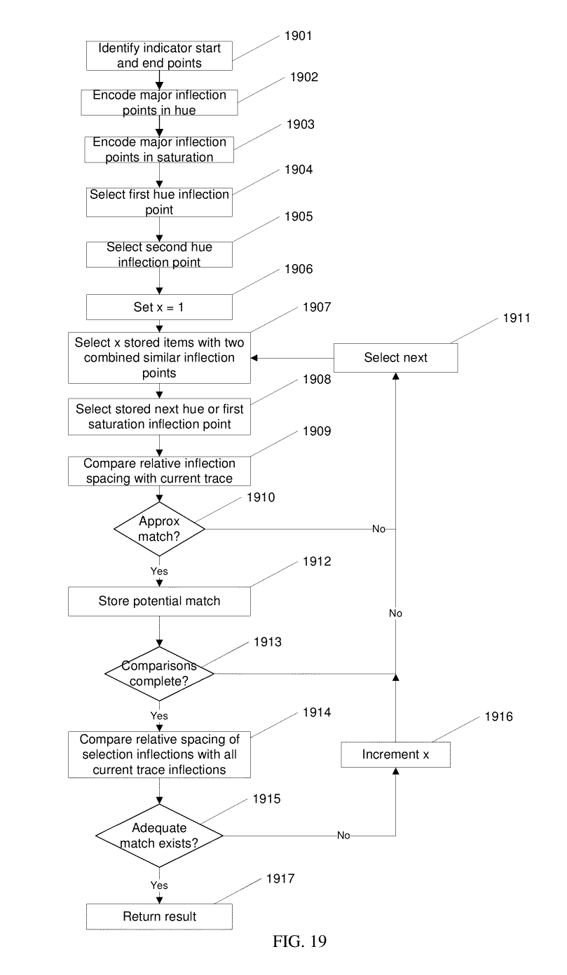

FIG. 19 shows a flow chart for a method of detecting the type of organism using the inflection points in the graphs of the culture medium expressed as a saturation/hue curve, since rather than being expressed in terms of the amount of red, blue and green in a colour a measurement can be expressed as the hue of that colour and the amount of saturation of that colour.

In FIG. 19 at 1901 the typical starting and ending points of the indicator colour for the target cultures are provided. Next at 1902 the inflection points in the hue which identify particular organisms of interest when grown in the culture with the indicator are encoded. Similarly at 1903 the inflection points in the saturation curve for these organisms are entered, and typically these are not coincident with those for hue.

At 1904 the first hue inflection point of a current growth curve is selected, and at 1905 the second hue inflection point. At 1906 the comparison enters a loop in which the selected inflection points are compared against the values for each of the stored organisms by selecting at 1907 from any matches the next inflection in either hue or saturation at 1908 and comparing the relative time difference of this third point relative to the first two at 1909. If an approximate match is found at 1910 the match is stored as a potential match at 1912. If the comparisons are not completed at 1913 the relative spacings of the selected inflections is compared with known trace inflections for a merely adequate match at 1915, and if one exists it is marked and the comparisons continued until the selection is exhausted. For each potential match found the spacing of all inflections is compared with the current culture under examination at 1914 and where a match is found a result is returned at 1917. If no match is found a further classification process (not shown) utilising combinations of the organisms in similar manner to that of FIG. 15 is begun in order to identify cultures which contain a mix of targeted organisms.

Detection of the flexion and inflexion points in the curves of light transmission and reflection may be mathematically quantified to allow automatic detection of the points on the curves which can act as indicators for the organism(s) which are present.

Detection of the times at which this occurs in relation to the overall growth curve, in combination with irregularities in the change of hue provide a distinctive signature which is amenable to solution.

### Growth Media

The samples are grown in a medium which has been standardised and calibrated against known organisms. Preferably the growth medium is held in a dry state in a rupturable container or pouch inside a sterile transparent plastics container of the type described in PCT/NZ2005/000139. Suitable growth media are described below. Whatever growth media is chosen it should be constant across a series of standard test containers and calibrated against known organisms so that the graphs shown above can be reliably used to ascertain the identity of micro-organisms from an environmental sample by comparing the growth pattern with the pre-calibrated graphs and machine readable data. To ensure reliable detection of a class of organisms, all the parameters (media, type of container, light source, optical filters or wavelengths chosen, incubation temperature, and the volume of the liquid sample) should be kept constant so that the only variable is the identity and quantity of the micro-organisms in the sample.

It should be noted, that these growth media compositions may be altered to allow for the specific growth and detection of various micro-organisms; the present examples relate to the detection of coliform bacteria (Table 1 and Table 2) and staphylococcal bacteria (Table 3).

### Example 1

### Coliform Bacteria

One preferred growth media composition contains an indicator (e.g. Resazurin), an inhibitor (e.g. Bile Salts) of non-specific micro-organisms (i.e. those micro-organisms not being tested for), a protein source (e.g. Casitone—pancreatic digest of casein), an energy source (e.g. lactose), and at least one salt (e.g. NaCl). An example of this composition is described below; Table 1. In addition, a range of the percentage of each component of the composition has been given within which the detection of coliforms may still be carried out under the present invention. Another growth medium composition suitable for testing for the presence of coliform bacteria (including E. coli) is given in Table 2.

TABLE 1

| Component | Percentage (w/v) | Range (% w/v) |
|---|---|---|
| Lactose | 3 | 0.5-10 |
| Bile Salts | 0.44 | 0.1-1.0 |
| Casitone | 0.2 | 0.02-1.0 |
| NaCl | 0.643 | 0.4-1.0 |
| KCl | 0.037 | 0.01-0.06 |
| CaCl2—H2O | 0.0184 | 0.01-0.03 |
| MgCl2—6H2O | 0.0091 | 0.005-0.02 |
| Resazurin* | 0.00058 | 0.0001-0.01 |

*Resazurin is a redox dye indicator—other similar indicators could be used—for example Triphenyltetrazolium chloride (TCC) and Rosolic Acid (Table 3)
*Resazurin is also known as (7-Hydroxy-3H-phenoxazin-3-one 10-oxide) Sodium Salt and can be purchased from Sigma as Resazurin R-2127.

TABLE 2

| Component | Percentage (w/v) |
|---|---|
| Lactose | 1 |
| Bile Salts | 0.44 |
| Casitone | 0.2 |
| NaCl | 0.643 |
| KCl | 0.037 |
| CaCl2—H2O | 0.0184 |
| MgCl2—6H2O | 0.0091 |
| Resazurin* | 0.00058 |

### Example 2

### Staphylococcal Bacteria

An example of the range of components used in a growth medium composition which may be used to support the growth and detection of staphylococcal bacteria is given below in Table 3.

TABLE 3

| Component | Percentage Range (w/v) |
|---|---|
| Bacto tryptone | 0.2-5.0 |
| Yeast extract | 0.1-0.5 |
| Lactose | 0.1-0.5 |
| Mannitol | 0.5-2.0 |
| dipotassium phosphate | 0.2-1.0 |
| Sodium Chloride | 5.0-10.0 |
| Rosolic Acid | 0.2-5.0 |

It should be noted that the components of the composition of Table 1, 2 or 3 may be varied depending on the type of tests to be carried out. Where meat samples are to be tested the composition of Table 1 preferably contains no salt. In addition, alternative redox dyes or other indicators may be utilised. Further, the protein, salt, and energy sources of the compositions herein described may be altered to support the growth of various alternative micro-organisms.

The calibration graphs illustrate the growth (and death) patterns for coliform bacteria including specific E. coli species grown in vials or other containers containing a standard growth media (standard in the sense that a set of containers for testing environmental samples for the presence or absence of coliforms would contain identical amounts of the identical growth media composition (whatever is used as the reference media for the calibration graphs for that family of micro-organism).

The following description will describe the invention in relation to preferred embodiments of the invention, namely a testing container for assisting a user in manually taking and/or testing a sample. The invention is in no way limited to these preferred embodiments as they are purely to exemplify the invention only and it is noted that possible variations and modifications are readily apparent without departing from the scope of the invention.

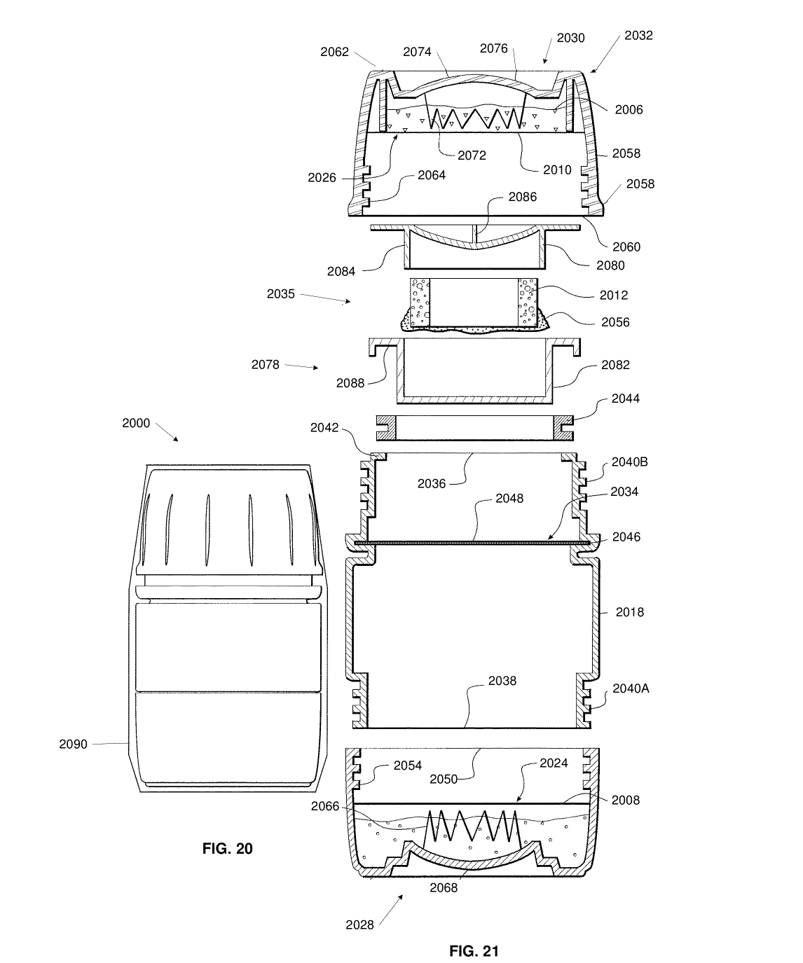

Turning to FIGS. 20-22, one embodiment of a container 2000 formed in accordance with the present invention is shown. Generally described, the container 2000 may be used to take a sample and analyse the sample for the presence of an analyte of interest. Moreover, the container is adapted to store a liquid 2002 within the container and two additives 2004 and 2006. The first additive 2004 is a reagent additive used to facilitate the testing of the contents of the container 2000 for the presence of an analyte of interest. The second additive 2006 is a prophylactic additive used to reduce the hazards presented by the contents of the container 2000 after testing has occurred. The liquid 2002 is separated from each of the additives 2004 and 2006 by two barriers 2008 and 2010. The barriers 2008 and 2010 may be selectively compromised by the user to permit the liquid 2002 to mix with the additives 2004 and 2006.

Disposed inside of the container 2000 is a sample collection member 2012 that may be removed from the container 2000 and used to collect a sample which potentially may contain the analyte of interest. Once the sample is collected, the sample collection member 2012 is returned to the container and intermixed with the liquid 2002, in conjunction with the reagent additive 2004 which is released via the compromising of the first barrier 2008. The contents of the container are then tested. Once testing is complete, the prophylactic additive 2006 is released via compromising of the second barrier 2010 to reduce the hazard presented by the contents, permitting the container and its contents to be disposed of with reduce risk of harm to humans or the environment.

In light of the above general description of the container 2000, the structure of the container 2000 will now be described in greater detail. The container 2000 may include a vessel 2014 capable of storing a substance 2016 (i.e. the contents of the container) in a leak proof environment. The vessel 2014 may be subdivided into a main compartment 2018, a first auxiliary compartment 2020, a second auxiliary compartment 2022, a first barrier assembly 2024, a second barrier assembly 2026, a first release assembly 2028, a second release assembly 2030, a cap assembly 2032, a filter assembly 2034, and a sampling assembly 2035. Each of these assemblies will now be described in detail.

The main compartment 2018 may be a generally cylindrically shaped structure having a first open end 2036 and a second open end 2038. The main compartment may be made of any suitable rigid or semi-rigid material, one suitable example being plastic, and is preferably made of a transparent or semi-transparent material. The main compartment 2018, when the first and second open ends 2036 and 2038 are blocked off, is adapted to hold a predetermined quantity (volume) of liquid 2002 without leaking. Although this amount of liquid may be any desired amount, in the illustrated embodiment, the predetermined volume of liquid 2002 is between about 50 ml and 1 litre, preferably between 100 and 500 ml, and most preferably about 120 ml.

Preferably the first and second open ends 2036 and 2038 each include attachment assemblies 2040A and 2040B for permitting the attachment, in either a permanent manner, or a removable manner, of the cap assembly 20132 and the first auxiliary compartment 2020 to the main compartment 2018. The attachment assemblies 2040A and 2040B may include threads, snap fit connections, quick-to-connect fittings, friction fit connections, and/or fasteners to permit the coupling of the cap assembly 2032 and the first auxiliary compartment 2020 to the main compartment 2018.

The first open end 2036 may include a lip 2042. The lip 2042 is preferably inward facing and annular in shape. The lip 2042 may be used to receive and retain a seal 2044 in place, the purpose of which will be described in greater detail below. Further, the main compartment 2018 may include an annular channel 2046. The annular channel 2046 may be used to receive and retain a filter 2048, the purpose of which will also be described in greater detail below.

The first auxiliary compartment 2020 may be a substantially cylindrical structure having an open end 2050 and a closed end 2052. The open end 2050 preferably includes an attachment assembly 2054 for cooperatively engaging and attaching to the attachment assembly 2040A associated with the main compartment 2018. Preferably the attachment assembly 2054 is adapted to non-removeably attach the auxiliary compartment 2020 to the main compartment 2018, although it is noted that it alternatively may removeably attach the auxiliary compartment 2020 to the main compartment 2018. The attachment assembly 2054 may include threads, snap fit connections, quick-to-connect fittings, friction fit connections, adhesives, and/or fasteners to permit the coupling of the cap assembly 2032 and the auxiliary compartment 2020 to the main compartment 2018.

The auxiliary compartment 2012 is adapted to store the reagent additive 2004 therein. The reagent additive 2004 may be any substance or compound, whether a liquid, solid, gas, or combination thereof. Preferably the reagent additive 2004 is a solid, and most preferably is a substantially dry powder. The reagent additive 2004 may be any substance or compound that provides at least a perceived benefit in the testing of the contents 2016 of the container for the presence of the analyte of interest. A few suitable examples of a reagent additive 2004 are a dye, an acid, a base, a chelating agent, a pH indicator, a growth media, oxidant, reductant, etc.

The cap assembly 2032 preferably includes a cap 2058. The cap 2058 may be a substantially cylindrical structure having an open end 2060 and a closed end 2062. The open end 2060 may include an attachment assembly 2064 for removeably coupling the cap assembly 2032 to the main compartment 2018. The attachment assembly 2064 may comprise threads, snap fit connections, quick-to-connect fittings, friction fit connections, and/or fasteners to permit the coupling of the cap assembly 2032 to the main compartment 2018.

The first barrier assembly 2024 is preferably disposed in the first auxiliary compartment 2020. The first barrier assembly 2024 may at least partially and preferably fully define the boundary between the main compartment 2018 and the first auxiliary compartment 2020. The first barrier assembly 2024 includes a barrier 2008 which may be used to seal off the second open end 2038 of the main compartment 2018. The barrier 2008 is preferably impermeable and water proof. The first barrier 2008 may be attached to the end of the main compartment 2018 by any suitable means, one example being by adhesive as is done in the illustrated embodiment, and another example being by mechanical means or fasteners. The first barrier 2008 is preferably designed to be selectively compromised, such as by rupturing or piercing, or by providing a mechanical opening (one example being rotation of the barrier to align apertures in the barrier with other apertures), to permit the contents 2002 of the main compartment 2018 to mix with the contents of the auxiliary compartment 2020. The barrier 2008 may be permanently installed in the container 2000.

The second barrier assembly 2026 is preferably disposed in the cap 2058. The second barrier assembly 2026 at least partially and preferably fully defines the boundary between the main compartment 2018 and the second auxiliary compartment 2022 and their respective contents. The second barrier assembly 2026 includes a second barrier 2010 which may be used to seal off the first open end 2036 of the main compartment 2018. The second barrier 2010 is preferably impermeable and water proof. The second barrier 2010 may be attached to the cap 2058 by any suitable means, one example being by adhesive as is done in the illustrated embodiment, and another example being by mechanical means or fasteners. The second barrier 2010 is preferably designed to be selectively compromised, such as by rupturing or piercing, or by providing a mechanical opening (one example being rotation of the barrier to align apertures in the barrier with other apertures), to permit the contents of the main compartment 2018 to mix with those of the second auxiliary compartment 2022. The second barrier 2010 may be permanently installed in the container 2000.

The first release assembly 2028 is designed to be activated to compromise the waterproof integrity of the first barrier 2008 and thus, initiate the mixing of the contents of the compartments 2018 and 2020. In the illustrated embodiment, the release assembly 2028 is disposed within the first auxiliary compartment 2020. The release assembly 2028 may include one or more piercing members, such as the teeth 2066 illustrated, which may be moved from a retracted position (shown in solid lines) to an extended position (shown in phantom lines in FIG. 5) where the piercing members compromise the first barrier 2008 by piercing the first barrier. The piercing members 2066 are preferably disposed on a resilient member 2068 which may be flexed by the user, preferably by hand or finger pressure, to transition the piercing members between their retracted and extended positions. When the pressure is released, the resilient nature of the resilient member 2068 automatically transitions the piercing members 2066 back to their retracted position. The resilient member 2068 is preferably located in a recess 2070 in the container 2000 such that the resilient member 2068 is out of the way and not likely to be inadvertently or accidentally transitioned into its extended position.

The sampling assembly 2035 may be disposed in the cap assembly 2032. The sampling assembly 2035 may include a sample collection member 2012. The sample collection member 2012 may be any device useful in collecting a sample 2056. A few suitable examples of sample collection devices 2012 are swabs, sponges, pads, and liquid collection devices (i.e. small test tubes, scoops, eye droppers, etc.). In the illustrated embodiment, the sample collection member 2012 is a sponge of a non-natural (synthetic) nature. The illustrated collection member 2012 may take any shape, one suitable example being the hollow cylindrical shape illustrated. The sample collection member 2012 may be adapted to be removed from the container and used to collect a sample 2056 for testing, and replaced in the container 2000 for testing of the sample collected, as will be described in greater detail below.

The sampling assembly 2035 may include a sample collection member housing assembly 2078 disposed in the container 2000, the sample collection member housing assembly 2078 adapted to house the sample collection member 2012. The sample collection member housing assembly 2078 may include at least a first portion 2080 and a second portion 2082 adapted to sealingly engage each other to fully encompass the sample collection member 2012 in a sealed/enclosed environment.

The first portion 2080 may be adapted to removeably hold the sample collection member 2012. More specifically, first portion 2080 may include an attachment structure 2084 that removeably holds the sample collection member 2012 to the first portion 2080. Preferably, the attachment structure 2084 holds the sample collection member 2012 to the first portion 2080 such that the sample collection member 2012 can be released without the user having to touch the sample collection member 2012 and potentially contaminating the sample collection member 2012. In the illustrated embodiment, the attachment structure 2084 is in the form of a hollow cylindrical structure that is sized and configured to receive the sample collection member 2012 in a friction fit/interference fit manner.

The first portion 2080 may include a graspable member 2086 which is adapted to be grasped by a user. The graspable member 2086 is adapted to be grasped by the user to permit the user to remove the first portion 2080 from the container 2000, contact a surface or substance to be sampled with the sample collection member 2012, and replace the sample collection member 2012 in the container by brushing the collection member 2012 against the seal 2044 to knock the sample collection member 2012 off of the attachment structure 2084. The sample collection member 2012 then falls into the container 2000. The user never needs to touch the sample collection member 2012 thereby reducing the risk the sample collection member 2012 is contaminated by the user's touch.

The second portion 2082 may include a sealing surface 2088 or flange adapted to sealingly engage the seal 2044. The second portion 2082 thereby serves to at least partially and preferably fully seal off the main compartment 2010, thereby keeping the container dry above the height of the meeting of the seal 2044 with the second portion 2082. The second portion 2082 may be removeably disposed in the end of the main compartment 2018, and may be held in position by any suitable means, one example being by compressing the second portion 2082 into the seal 2044 by a compressive force applied by the cap 2058.

The second release assembly 2030 is designed to be activated to compromise the waterproof integrity of the second barrier 2010 and thus, initiate the mixing of the contents of the compartments 2018 and 2022. In the illustrated embodiment, the second release assembly 2030 is disposed within the second auxiliary compartment 2022 and the cap 2058. The second release assembly 2030 may include one or more piercing members, such as the teeth 2072 shown, which may be moved from a retracted position (shown in solid lines) to an extended position (shown in phantom lines in FIG. 7) where the piercing members compromise the second barrier 2010 by piercing the barrier 2010. The piercing members 2072 are preferably disposed on a resilient member 2074 which may be flexed by the user, preferably by hand or finger pressure, to transition the piercing members between their retracted and extended positions. When the pressure is released, the resilient nature of the resilient member 2074 automatically transitions the piercing members 2072 back to their retracted position. The resilient member 2074 is preferably located in a recess 2076 in the cap 2058 such that the resilient member 2074 is out of the way and not likely to be inadvertently or accidentally transitioned into its extended position.

Turning to FIG. 20, preferably the container 2000 includes a protective layer 2090. The protective layer 2090 preferably encases most if not all of the container 2000. The protective layer 2090 may provide a barrier to the introduction of bacteria into the container 2000 and onto the outer surface of the container 2000. The protective layer 2090 is preferably transparent or semi-transparent. The protective layer 2090 is preferably adapted to be removed by the user ripping off the protective layer 2090. In one embodiment, the protective layer 2090 is a shrink wrap layer. Preferably, the protective layer 2090 hermetically seals the container within the protective layer 2090. Preferably, during manufacture, the protective layer 2090 is placed on the container 2000, and then the container 2000 is treated to kill germs, bacteria and the like, thus sterilizing the container 2000 and its contents. For instance, the container 2000 may be irradiated to kill micro-organisms, bacteria, germs, and the like present on or in the container 2000.

Preferably the container 2000 is subject to a predetermined minimum amount of ionizing radiation to sterilize the container. The predetermined minimum amount of ionizing radiation depends on how “clean” the manufacture wishes the container to be and is preferably greater than about 1, 2, 5, 10, or 20 kGy. Most preferably, more than about 20 kGy is used.

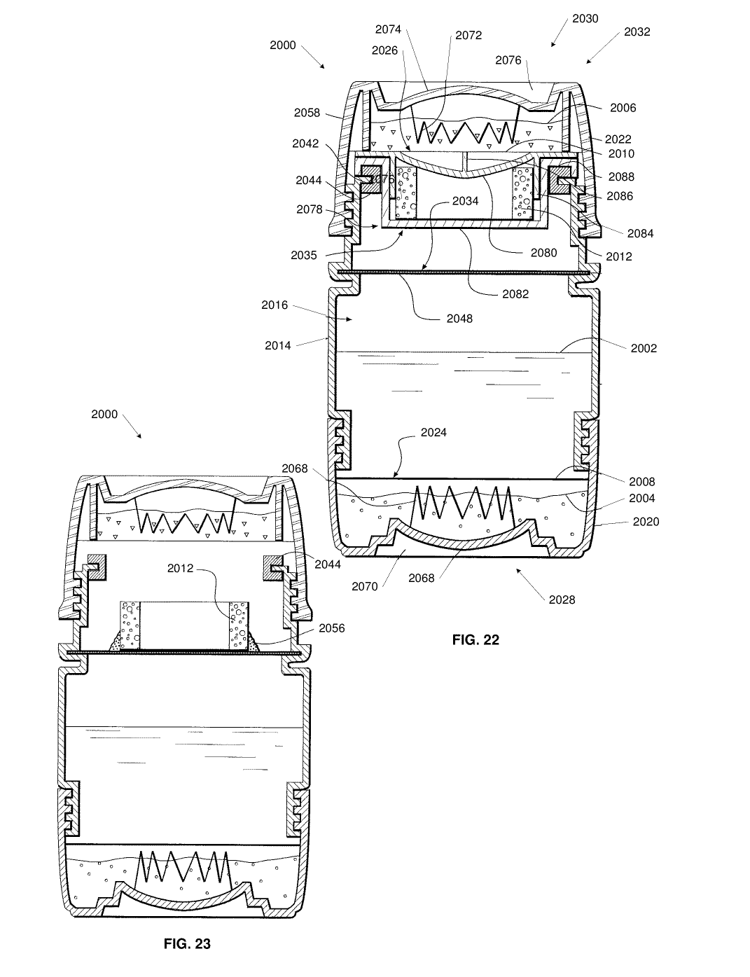

Referring to FIG. 20, in light of the above description of the structure of the container 2000, the use of the container 2000 will now be described. First, the user removes the protective layer 2090 by ripping the layer off and discarding same. Turning now to FIGS. 21, 22, the user then removes the cap 2058 and removes the first portion 2080 of the sample collection member housing assembly 2078 by grasping the graspable member 2086. If the second portion 2082 of the sample collection member housing assembly 2078 was not removed with the first portion 2080, the user reaches and removes the second portion 2082 from the container. The sample collection member 2012, as noted above, is removeably attached to the first portion, and remains attached to the first portion 2080 when the first portion 2080 is removed from the container 2000. Still holding the graspable member 2086, the user places the sample collection member 2012 in contact with a sample 2056 potentially containing the analyte of interest, such as by wiping the sample collection member 2012 upon a surface.

Turning to FIG. 23, once the sample 2056 is obtained, the sample collection member 2012 is returned to the container 2000. Preferably, the sample collection member 2012 is released from the first portion 2080 (See FIG. 3) without requiring the user to touch the sample collection member 2012, one suitable method involving brushing the sample collection member 2012 against the seal 2044 to knock the sample collection member 2012 free of its friction hold to the first portion, such that the sample collection member 2012 drops by gravity into the container 2000.

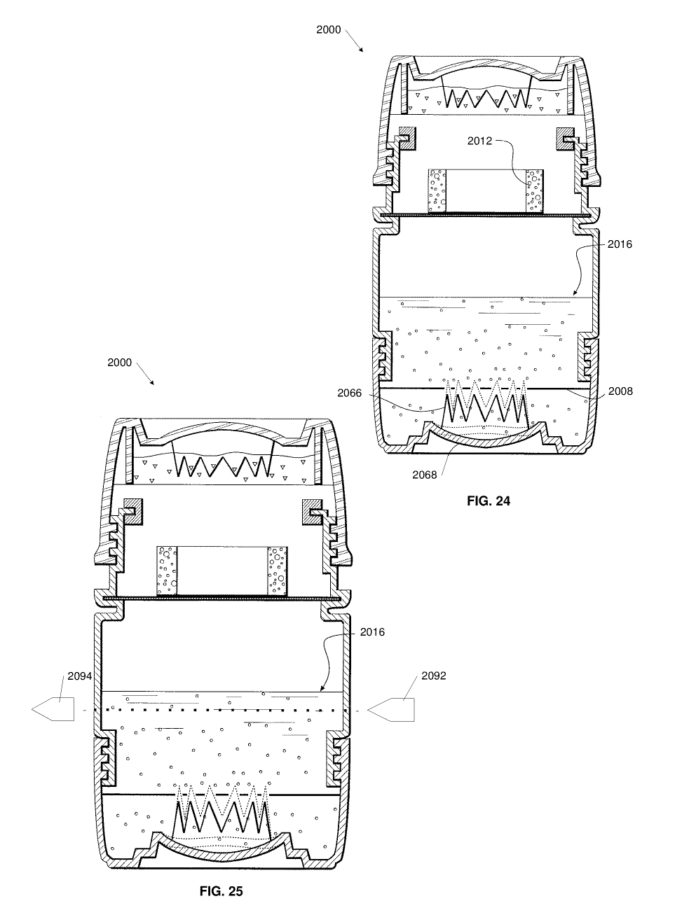

Turning to FIG. 24, the user presses on the resilient member 2068 to cause the piercing members 2052 to pierce the barrier 2008. The compromising of the barrier 2008 permits the contents of the first auxiliary compartment 2020 to mix with the contents of the main compartment 2018 to form a mixture 2016. The mixing of the contents may be assisted by the user shaking the container 2000. Further, the shaking causes the mixture 2016 to come in contact and mix with the sample present on the sample collection member 2012.

Referring to FIG. 25, once the mixing process has been completed and the sample is completely mixed with the mixture 2016, the testing of the mixture 2016 for the presence of the analyte of interest may commence. Although the mixture 2016 may be removed for testing, preferably the mixture 2016 remains in the container 2000 and is tested in situ. For instance, the mixture 2016 may be tested in situ for observable colour changes, formation of a participate, or for a change in a predetermined amount of absorption, reflection, or scattering of electromagnetic radiation. For instance, an electromagnetic radiation source 2092 may be used to emit a predetermined amount of electromagnetic radiation through the mixture 2016. A sensor 2094 may be used to determine the amount of radiation lost during the travel of the electromagnetic radiation through the container 2000. Depending upon the amount of radiation lost, the presence, or lack thereof, of the analyte of interest in the mixture 2016 may be determined.

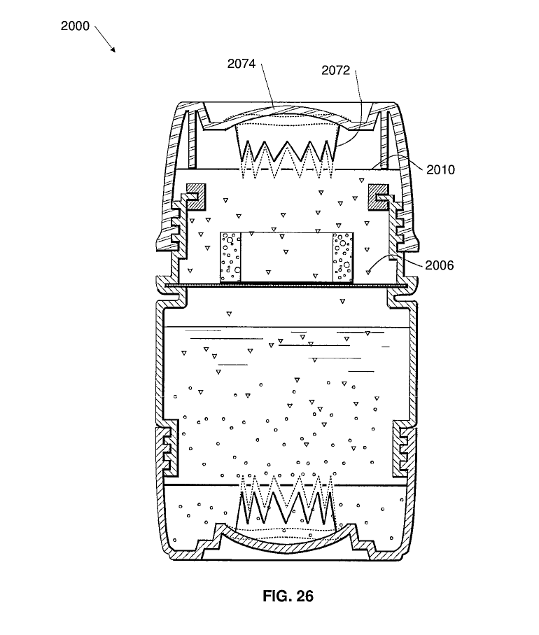

Turning now to FIG. 26, the user may press on the resilient member 2074 to cause the piercing members 2072 to pierce the barrier 2010. The compromising of the barrier 2010 permits the contents of the second auxiliary compartment 2022 to mix with the contents of the main compartment 2018. The mixing of the contents may be assisted by the user shaking the container 2000.

Preferably, the second auxiliary compartment 2022 is filled with a prophylactic additive 2006 that reduces the potential of the mixture 2016 to harm the environment or human health, to among other things, facilitate the disposal of the container 2000. For instance, as described more fully above, the prophylactic additive 2006 may be a disinfectant that kills any harmful bacteria that may be present in the sample, making the container 2000 suitable for disposal as non-hazardous waste. Alternately, the additive 2006 may be a second reagent needed to be added to the mixture 2016 to facilitate testing of the mixture 2016.

Of note, preferably the contents of the container 2000 are not mixed nor the protective layer 2090 removed, until just prior to sample collection. Accordingly, by following this procedure, and sterilizing the container at the end of the manufacturing process, the container 2000 can have an extended shelf life which preferably spans greater than a predetermined duration, a few suitable examples being greater than one year, two years, three years, four years, five years, six years, and even as much as greater than seven years.

In some instances it may be preferred to accommodate more than one sample within a container, perhaps because a smaller sample will still allow detection of contamination at the level required, perhaps because exactly identical development times are required for a comparison.

In such a case an insert capable of maintaining several samples separate through incubation is required, while yet allowing all of the cultures to be easily sterilized at the end of process.

### Advantages

A container formed in accordance with the present invention will exhibit one or more of the following advantages:

Increased shelf life;

### Sterile;

Contains safe, substantially contaminate free or sterile water;
Easy to use;
Less expensive;
Reduced potential of contaminates being introduced into the container during sampling or testing;
Reduced potential that the contents of the container will cause harm to humans or the environment after sampling or testing is complete;
Container can be disposed of as non-hazardous waste;
Reduced handling and opening of the container required during testing;
Less exposure to handlers to the contents of the container;

### Reliable; and

Easy to manufacture.

While the invention is described in relation to water testing the invention is suitable for any material which may be dissolved, suspended or otherwise cultured in any fluid. Thus in the testing of crustacean a shellfish may be added whole or macerated to the water with an additional growth medium and indicator for the micro-organism concerned if desired. In the testing of milk powder the powder may be dissolved in water with added growth adjuvant and an indicator.

The apparatus itself may be readily portable and self-contained, and this together with the comparatively rapid response allows its use in situations where a normal laboratory result would be too slow to be useful, as for instance in determining whether flooding has caused a pathogen problem. For use in bulk testing situations an apparatus containing multiple vials, each with a light source and detector, may be provided. The outputs are sequentially monitored by a processing and recording apparatus.

While the optical system described provides white light and is sensitive in only three colour bands it is possible to substitute a system in which a variable narrow band filter is applied to either the source or the detector, allowing a continuous scan across the ultra-violet, visible, and infra-red spectrum.

Those skilled in the art will appreciate that in order to test for other micro-organisms that other growth media would have to be used and calibrated against known species of micro-organisms. Those media would contain reagents that favoured the growth of the micro-organisms to be tested but selectively inhibit the growth of other micro-organisms. (This is the function of the bile salts in the medium of Example 1).

It is to be understood that even though numerous characteristics and advantages of the various embodiments of the present invention have been set forth in the foregoing description, together with details of the structure and functioning of various embodiments of the invention, this disclosure is illustrative only, and changes may be made in detail so long as the functioning of the invention is not adversely affected.

For example the particular elements of the mechanism for measuring the optical reflection from the medium and contents may vary dependent on the particular application for which it is used without variation in the spirit and scope of the present invention.

Similarly the wave bands of light which illuminate the medium for the measurements may be white light with colour sensitive sensors or they may be sequentially applied bands of different coloured light with wideband sensors.

In addition, although the preferred embodiments described herein are directed to the measurement of micro-organism growth curves using multiple wave bands of transmitted light and a single reflected light band, it will be appreciated by those skilled in the art that the teachings of the present invention can be applied to other systems such as those using multiple differing bands for the reflected light and a single band for transmitted light, without departing from the scope and spirit of the present invention.

In addition, although the preferred embodiments described herein are directed to analysis of micro-organisms in an estuarine system, it will be appreciated by those skilled in the art that the teachings of the present invention can be applied to other systems such as water quality monitors, without departing from the scope and spirit of the present invention.

For instance the system may be adapted to provide checks throughout the supply chain of any perishable foodstuff. In such instances the foodstuff is handled as batches and samples are taken at each step in which the foodstuff batch is handled. Thus samples may be taken of all the inputs to the primary collection point (for instance for a seafood supplier of oysters: of the storage or wash water and the raw oyster flesh at packing), at the secondary distributor (if a batch is broken: swabs of the new container, of the decanted oyster fluid), at the final destination (at a hotel: sample of the oyster fluid as decanted, swab of the storage environment).

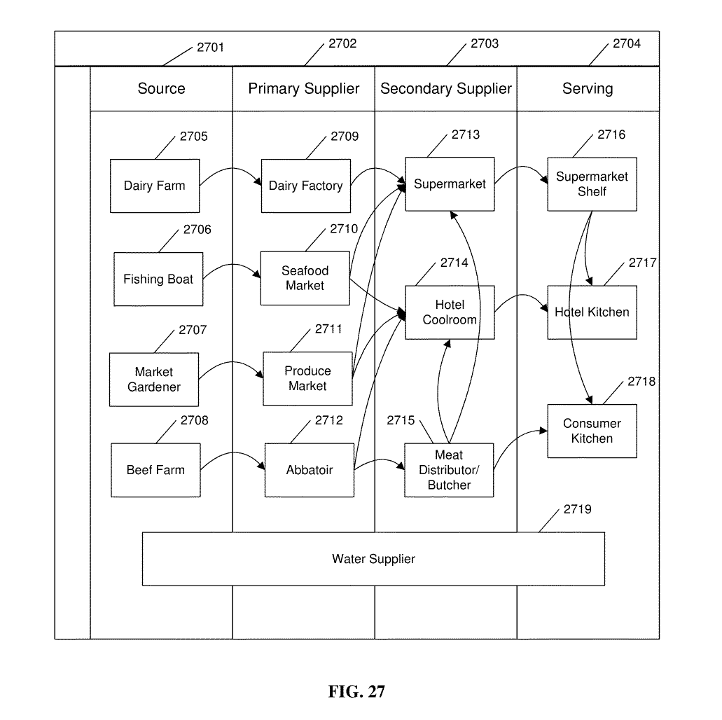

FIG. 27 shows at 2701 a column representing typical sources of perishable foodstuffs, at 2702 the principal primary source of supplies, at 2703 a column of some secondary suppliers and at 2704 a column of some typical servers of the foodstuffs.

The source may, for instance, be a dairy farm 2705 supplying milk, a fishing boat 2706 supplying fish, oysters or squid, a market gardener 2707 supplying vegetable or a beef farm 2708 supplying beef or veal. The sources send the foodstuffs to what can be considered the primary source of supply such as a dairy factory 2709 supplying milk, cream or cheese, a seafood market 2710 supplying fish, surimi or smoked salmon, a produce market 2711 supplying potatoes, lychees or salad vegetables and an abbatoir 2712 supplying lamb, beef or alpaca carcases.

The product is from the primary supplier is bought by a secondary supplier such as a supermarket 2713, a hotel 2714, which may store the product in their cool room, a meat distributor or butcher at 2715.

The end product may be purchased from a supermarket shelf at 2716 and might be prepared in a hotel kitchen at 2717 or a consumer kitchen at 2718.

Shown at the bottom of the diagram is the water supplier 2719. The water supply is normally a critical factor at every stage since products may be washed or sprayed at several points in their travel or preparation. Continual tests of water quality at each stage may be essential element in a train of causation.

Naturally it will probably be impossible to culture a sample before the next step in the chain takes place, but at each stage the results of each test, identified against the batch number and by the type of test, are transferred to a central database. The central database is itself traversed by an application identifying those samples which exceed a maximum contamination level and can provide alerts to the supplier of the test results, the occupant of the tested location and any appropriate authorities.

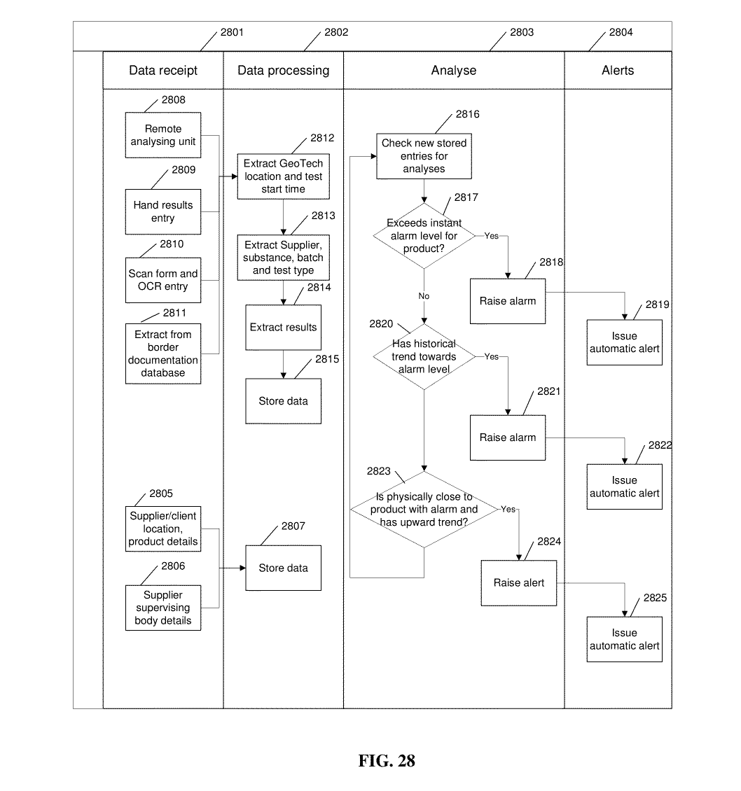

FIG. 28 shows how the results may be collated into a database and may automatically generate an alarm if contamination levels rise towards a danger point. The processing includes four phases, the data receipt at 2801, the data processing at 2802 which results in the storage of information in a database, and the continual analysis process of entries at 2803 and the issuing of alerts at 2804.

Information on supplier or client such as their physical location, their contact, the details of the products they sell the level of alarm which should be provided if contamination is found and any other relevant details are entered at 2805. At 2806 are entered the details on the body or bodies which supervises the quality of the product or products which the supplier or client provides. This may for instance be a government body, a local body, a contracted authority or some other regulative authority. This information is then stored at 2807.

At 2808 the results from a remote self-reporting analysis unit may be received and automatically entered, at 2809 results may be hand entered at a call centre or an office, at 2810 the data on a known form may be scanned and OCR'd for entry and at 2811 the data from a cross-border database or other documentation database capturing information equivalent to a form may be interrogated and extracted. Such documentation may take the form proposed by UN/CEFACT (United Nations Centre for Trade Facilitation and Electronic Business)

The data received is processed by extracting or looking up at 2812 the GeoTech location of the test, the time the test was started, etc. At 2813 information on the supplier, the substance tests, the particular batch number concerned and the type of test is extracted and at 2814 the test results themselves are extracted. Once all the information is verified as correct the data received is stored in the database at 2815.

Once stored the information is subject to a continually running analysis process interrogating the data within the database. This process will, for instance, check at 2816 for newly stored data, check at 2817 that the batch results do not exceed the alarm level for that product, and if they do raise an alarm at 2818 which will result in the issuing of an automatic alert to the supplier and possibly to the regulatory authority concerned at 2819.

If no instant alarm is provided any previous analysis relating to that batch is checked at 2820 and any historical trend towards the alarm level as the batch has proceeded through the various supply points is identified. If the product trend is such that the next report on that batch is likely to exceed alarm levels then again an alarm is raised at 2821 and an automatic alert issued at 2822 to the supplier and possibly to the next destination for the batch if this is known from the information in the database.

If no such alarm is raised then at 2823 the product is checked for an unusual upwards trend level and physical proximity at one stage to other similar products which currently have an alarm indicated. This allows an alert to be raised at 2824 to allow an automatic alert at 2825 to a regulatory authority of possible future problems.

In this way a continual check may be kept on either a supplier or a particular batch of product from a supplier or a number of supplies in the same geographical area.

In the same way the results of tests for biological agents spread by other than foodstuffs may be made, for instance the system may be used for the detection and tracking of air spread or commerce spread pathogens and infections.

### Variations

It is to be understood that even though numerous characteristics and advantages of the various embodiments of the present invention have been set forth in the foregoing description, together with details of the structure and functioning of various embodiments of the invention, this disclosure is illustrative only, and changes may be made in detail so long as the functioning of the invention is not adversely affected. For example the particular manner of implementing the method may vary dependent on the particular application for which it is used without variation in the spirit and scope of the present invention.

Although the invention has been described with reference to a portable incubator suitable for carrying out the method of testing of this invention, where the incubator can be taken to different locations, and samples taken and analysed on the spot, it is equally applicable that the incubator could be positioned at a fixed location for example in a factory or food processing plant, and the various sample bottles taken at different points of the food processing plant, and then carried to the incubator, and incubated. In such a factory application, the data base may be operated by a stand alone computer connected to the incubator.

However in most cases it is preferable that the database is remote from the various incubators, so that a number of incubators and samples can be taken across a large geographic area, and the results can be mapped by suitable analysis of the data stored in the database.

By storing the data in a database, it is also possible to track the spread of micro organisms, over a spatial area, but it is also possible to track occurrences of micro organisms over time not only in particular areas, but to track mutations, and other changes to populations to micro organisms. Throughout the specification reference is made to location information and date and time stamps of samples, and will thus be appreciated that the database or mapping aspects of this invention effectively rely on coordinates in four dimensional space time, which can be analysed in different ways as outlined above.

In addition it should be noted that a separate aspect of this invention is the fact that the same sample containers and incubator can be used to detect different types of micro organisms in the same sample, and this differentiation of the detected micro organisms can be used with the mapping applications, but can also be used as a stand alone test to determine the different types of micro organisms detected at a particular site, and where time permits, the enumeration of this different micro organisms at that site.

Throughout the description of this specification, the word “comprise” and variations of that word such as “comprising” and “comprises”, are not intended to exclude other additives, components, integers or steps.

It will of course be realised that while the foregoing has been given by way of illustrative example of this invention, all such and other modifications and variations thereto as would be apparent to persons skilled in the art are deemed to fall within the broad scope and ambit of this invention as is hereinbefore described.

### INDUSTRIAL APPLICABILITY

The portable methods and apparatus of the invention are used in the testing of potentially harmful samples within the health industry. The present invention is therefore industrially applicable.

1. A portable micro-organism detection apparatus comprising:
an incubator at least partially surrounding a container receiving space capable of receiving a rigid substantially transparent fluid container with at least one light path through the container,
at least one light source mounted on or in an incubator capable of transmitting light into the fluid container,
at least one light sensor mounted on or in the incubator and capable of detecting light of at least one colour which has passed through at least part of the fluid from the at least one light source,
stored calibration information on a non-transient medium on the light changes over time measured in a similar container containing a sample of a known identified micro-organism,
location detection system connected to or mounted on the incubator to provide location data of where the sample was taken,
a micro-processor capable of controlling the operation of the incubator and the light source(s) and light sensor(s) and comparing, analyzing and storing the results, and receiving location data from the location detection system,
stored calibration information on a non-transient medium on the light changes over time measured in a similar container containing a sample of a known identified micro-organism,
the micro-processor having a comparator capable of detecting changes over time resulting from a sample in the rigid substantially transparent fluid container being incubated in the incubator, and comparing it with the stored calibration information to determine the presence or absence of a particular micro-organism,
a data logger to store the results of the comparison and a date and time stamp and location information from the location detection system.

2. Apparatus as claimed in claim 1, wherein the incubator has a transmitter to send the results to a base station.

3. A method of detecting micro-organisms from changes in the colour of a growth medium in which a sample potentially containing micro-organisms is immersed, by sealing a sample from a location in a rigid substantially transparent fluid container with at least one light path through the container, placing the container in an incubator, transmitting light into the fluid container over time from at least one light source mounted on or in an incubator; detecting light of at least one colour which has passed through at least part of the fluid from the at least one light source by at least one light sensor mounted on or in the incubator; sending data from the at least one light sensor to a micro-processor having a comparator which analyses the light changes over time resulting from a sample in the rigid substantially transparent fluid container being incubated in the incubator, by comparing the detected light changes with stored calibration information to determine the presence or absence of at least one particular micro-organism; and stores the results of the comparison for the sample in a data logger together with location information and a date and time stamp for that sample.

4. A method as claimed in claim 3 wherein the results are transmitted to a base station and stored in a database.

5. A method as claimed in claim 4 wherein a plurality of samples are analyzed and the results transmitted to the base station for analysis by a computer.

6. A method as claimed in claim 5 wherein rate of growth over time of the micro-organism(s) within the sample is assessed to enumerate the number of micro-organism(s) detected at the location.

7. A method as claimed in claim 5 wherein the computer analyses the occurrence or spread or decline of spatial populations of microbiological organisms from the sample data and location and date time stamp of each sample.

8. A method as claimed in claim 3 wherein the rate of growth over time is matched against stored calibration information for differing micro-organisms to determine the closest match.

9. A method of detecting micro-organisms as claimed in claim 3 wherein the growth medium is optimised for E. coli.

10. A method of detecting micro-organisms as claimed in claim 3 wherein the growth medium contains lactose and growth adjuvants.

11. A method as claimed in claim 6 wherein the computer analyses the occurrence or spread or decline of spatial populations of microbiological organisms from the sample data and location and date time stamp of each sample.
